(19) United States
(12) Patent Application Publication
(10) Pub. No.: US 2026/0165606 A1
(43) Pub. Date: Jun. 18, 2026

(54) FEATURE QUANTITY DATA GENERATION DEVICE, GAIT MEASUREMENT DEVICE, PHYSICAL CONDITION ESTIMATION SYSTEM, FEATURE QUANTITY DATA GENERATION METHOD, AND RECORDING MEDIUM

(71) Applicant: NEC Corporation, Minato-ku, Tokyo (JP)

(72) Inventor: Hiroshi KAJITANI, Tokyo (JP)

(73) Assignee: NEC Corporation, Minato-ku, Tokyo (JP)

(21) Appl. No.: 18/711,169

(22) PCT Filed: Dec. 10, 2021
(86) PCT No.: PCT/JP2021/045469

§ 371 Date: May 17, 2024

(51) Int. Cl. A61B 5/11

(52) U.S. Cl. CPC A61B 5/112

(57) ABSTRACT

Provided is a feature quantity data generation device that includes an acquisition unit that acquires time-series data of sensor data regarding movements of feet, a normalization unit that extracts gait waveform data for one gait cycle from the time-series data of the sensor data, and normalizes the extracted gait waveform data, an extraction unit that extracts, from the normalized gait waveform data, feature quantities related to a physical condition of an estimation target from gait phase clusters each constituted by one or more temporally consecutive gait phases, a selection unit that selects feature quantities to be used in estimating the physical condition from the extracted feature quantities for the respective gait phase clusters using a preset threshold as a reference, a generation unit that generates feature quantity data including the selected feature quantities, and an output unit that outputs the generated feature quantity data.

### TECHNICAL FIELD

The present disclosure relates to a feature quantity data generation device or the like that generates feature quantity data from data regarding a gait.

### BACKGROUND ART

With the growing interest in healthcare, services for providing information according to features (also referred to as a gait) included in a gait pattern have attracted attention. For example, a technique for analyzing a gait based on sensor data measured by a sensor mounted on footwear such as a shoe has been developed. Features of gait events related to a physical condition appear in time-series data of the sensor data. That is, if feature quantities of gait events can be accurately extracted, a physical condition can be estimated with high accuracy.

PTL 1 discloses a device that detects an abnormality of a foot based on a gait feature of a pedestrian. The device of PTL 1 extracts a characteristic gait feature quantity in a gait of a pedestrian wearing footwear, using data acquired from a sensor installed in the footwear. The device of PTL 1 detects an abnormality of a pedestrian who is walking while wearing the footwear based on the extracted gait feature quantity. For example, the device of PTL 1 extracts a feature site related to hallux valgus from gait waveform data for one gait cycle. The device of PTL 1 estimates a progress state of hallux valgus using a gait feature quantity of the extracted feature site.

PTL 2 discloses a system that calculates an index for analyzing a circumduction gait. The system of PTL 2 acquires information on an angular velocity of an ankle of a person during walking detected by a sensor attached to the ankle. The system of PTL 2 detects a swing phase, which is a time when a foot is off the ground, based on a change in angular velocity over time around a first axis that extends in a right-left direction of the person among the acquired information regarding the angular velocity. The system of PTL 2 specifies a predetermined time from the beginning of the swing phase as a time of interest. The system of PTL 2 calculates an index to be used in determining whether the person is performing a circumduction gait based on a change in angular velocity over time around a second axis that extends in a vertical direction within the time of interest among the acquired information regarding the angular velocity.

PTL 3 discloses a system that estimates a trip attribute of a terminal holder. The system of PTL 3 estimates a plurality of trip attributes defining a movement feature of a user who holds a mobile terminal, using global positioning system (GPS) data and acceleration sensor data of the mobile terminal. By using a result of estimating a first trip attribute included in the results of estimating the plurality of trip attributes and a result of estimating related information between the trip attributes, the system of PTL 3 corrects a result of estimating a second trip attribute different from the first trip attribute. For example, the system of PTL 3 clusters final positions of main trips in which a difference between an end time of a preceding main trip and a start time of a subsequent main trip is largest among a plurality of main trips in one day. The system of PTL 3 extracts a cluster having the largest number of elements, and estimates the coordinates of the center of gravity of the cluster as a position of a workplace.

### CITATION LIST

### Patent Literature

PTL 1: WO 2021/140658 A1
PTL 2: WO 2020/152817 A1
PTL 3: WO 2015/177858 A1

### SUMMARY OF INVENTION

### Technical Problem

In the method of PTL 1, a mid-stance period is detected using the gait waveform data of the plantar angle for two gait cycles, and gait waveform data for one gait cycle is generated based on the detected mid-stance period. That is, in the method of PTL 1, in order to generate gait waveform data for one gait cycle, the gait waveform data of the plantar angle for two gait cycles is required. In addition, in the method of PTL 1, the gait cycle of the gait waveform data is normalized based on time-series data on plantar angles. In the method of PTL 1, in order to normalize the gait cycle of the gait waveform data, time-series data on angular velocities detected by the sensor is integrated to generate time-series data on plantar angles. That is, in the method of PTL 1, several preprocessing stages are required in order to generate gait waveform data to be used in extracting a feature quantity to be used in estimating a physical condition.

In the method of PTL 2, a toe off or a heel contact can be detected based on temporal changes in angular velocity around a first axis. In PTL 2, normalization is performed in consideration of a gait speed of a patient, with a value obtained by dividing a difference (width) between a minimum value and a maximum value among angular velocities around a second axis by a maximum value among the angular velocities around the first axis being a feature quantity. PTL 2 does not disclose normalizing the time-series data on angular velocities in the time direction. Therefore, in the method of PTL 2, it is not possible to accurately detect a gait event other than the toe off or the heel contact from the temporal changes in angular velocity.

In the method of PTL 3, a position of a workplace or the like of a terminal holder can be estimated according to the number of components of a cluster generated based on position. PTL 3 does not disclose generating gait waveform data for one gait cycle or normalizing a gait cycle of gait waveform data. Therefore, in the method of PTL 3, it is not possible to detect a gait event for extracting a feature quantity to be used in estimating a physical condition.

An object of the present disclosure is to provide a feature quantity data generation device and the like capable of generating feature quantity data with which a physical condition can be estimated with high accuracy.

### Solution to Problem

A feature quantity data generation device according to one aspect of the present disclosure includes an acquisition unit that acquires time-series data of sensor data regarding movements of feet, a normalization unit that extracts gait waveform data for one gait cycle from the time-series data of the sensor data, and normalizes the extracted gait waveform data, an extraction unit that extracts, from the normalized gait waveform data, feature quantities related to a physical condition of an estimation target from gait phase clusters each constituted by one or more temporally consecutive gait phases, a selection unit that selects feature quantities to be used in estimating the physical condition from the extracted feature quantities for the respective gait phase clusters using a preset threshold as a reference, a generation unit that generates feature quantity data including the selected feature quantities, and an output unit that outputs the generated feature quantity data.

A feature quantity data generation method according to one aspect of the present disclosure includes acquiring time-series data of sensor data regarding movements of feet, extracting gait waveform data for one gait cycle from the time-series data of the sensor data, normalizing the extracted gait waveform data, extracting, from the normalized gait waveform data, feature quantities related to a physical condition of an estimation target from gait phase clusters each constituted by one or more temporally consecutive gait phases, selecting feature quantities to be used in estimating the physical condition from the extracted feature quantities for the respective gait phase clusters using a preset threshold as a reference, generating feature quantity data including the selected feature quantities, and outputting the generated feature quantity data.

A program according to one aspect of the present disclosure causes a computer to execute processing of acquiring time-series data of sensor data regarding movements of feet, processing of extracting gait waveform data for one gait cycle from the time-series data of the sensor data, processing of normalizing the extracted gait waveform data, processing of extracting, from the normalized gait waveform data, feature quantities related to a physical condition of an estimation target from gait phase clusters each constituted by one or more temporally consecutive gait phases, processing of selecting feature quantities to be used in estimating the physical condition from the extracted feature quantities for the respective gait phase clusters using a preset threshold as a reference, processing of generating feature quantity data including the selected feature quantities, and processing of outputting the generated feature quantity data.

### Advantageous Effects of Invention

According to the present disclosure, it is possible to provide a feature quantity data generation device and the like capable of generating feature quantity data with which a physical condition can be estimated with high accuracy.

### BRIEF DESCRIPTION OF DRAWINGS

FIG. 1 is a block diagram illustrating an example of a configuration of a gait measurement device according to a first example embodiment.

FIG. 2 is a conceptual diagram illustrating an example in which the gait measurement device according to the first example embodiment is arranged.

FIG. 3 is a conceptual diagram for explaining an example of a relationship between a local coordinate system and a world coordinate system set in the gait measurement device according to the first example embodiment.

FIG. 4 is a conceptual diagram for explaining human body planes to be used in the description regarding the gait measurement device according to the first example embodiment.

FIG. 5 is a conceptual diagram for explaining a gait cycle to be used in the description regarding the gait measurement device according to the first example embodiment.

FIG. 6 is a graph for explaining an example of time-series data of sensor data measured by the gait measurement device according to the first example embodiment.

FIG. 7 is a diagram for explaining an example of normalization of gait waveform data extracted from the time-series data of the sensor data measured by the gait measurement device according to the first example embodiment.

FIG. 8 is a graph for explaining another example of time-series data of sensor data measured by the gait measurement device according to the first example embodiment.

FIG. 9 is a diagram for explaining another example of normalization of gait waveform data extracted from the time-series data of the sensor data measured by the gait measurement device according to the first example embodiment.

FIG. 10 is a conceptual diagram for explaining an example of a gait phase cluster from which a feature quantity data generation device of the gait measurement device according to the first example embodiment extracts feature quantities.

FIG. 11 is a table relating to a specific example of a gait phase cluster from which the feature quantity data generation device of the gait measurement device according to the first example embodiment extracts feature quantities.

FIG. 12 is a graph for explaining an example in which the feature quantity data generation device of the gait measurement device according to the first example embodiment extracts a feature quantity of a gait phase cluster.

FIG. 13 is a graph for explaining an example of a correlation coefficient between a feature quantity of a gait phase cluster extracted by the feature quantity data generation device of the gait measurement device according to the first example embodiment and a value related to a physical condition of an estimation target.

FIG. 14 is a graph for explaining an example of verification of significance of the correlation between the feature quantity of the gait phase cluster extracted by the feature quantity data generation device of the gait measurement device according to the first example embodiment and the value related to the physical condition of the estimation target.

FIG. 15 is a graph for explaining an example of a correlation between a feature quantity of a gait phase cluster extracted by the feature quantity data generation device of the gait measurement device according to the first example embodiment and a value related to a physical condition of an estimation target.

FIG. 16 is a graph for explaining another example of a correlation between a feature quantity of a gait phase cluster extracted by the feature quantity data generation device of the gait measurement device according to the first example embodiment and a value related to a physical condition of an estimation target.

FIG. 17 is a flowchart for explaining an example of an operation of the feature quantity data generation device included in the gait measurement device according to the first example embodiment.

FIG. 18 is a block diagram illustrating an example of a configuration of a physical condition estimation system according to a second example embodiment.

FIG. 19 is a block diagram illustrating an example of a configuration of an estimation device included in the physical condition estimation system according to the second example embodiment.

FIG. 20 is a block diagram illustrating an example in which the estimation device included in the physical condition estimation system according to the second example embodiment estimates a physical condition.

FIG. 21 is a flowchart for explaining an example of an operation of the estimation device included in the physical condition estimation system according to the second example embodiment.

FIG. 22 is a conceptual diagram for explaining an example in which the second example embodiment is applied.

FIG. 23 is a block diagram illustrating an example of a configuration of a machine learning system according to a third example embodiment.

FIG. 24 is a block diagram illustrating an example of a configuration of a machine learning device included in the machine learning system according to the third example embodiment.

FIG. 25 is a conceptual diagram for explaining an example of machine learning by the machine learning device included in the machine learning system according to the third example embodiment.

FIG. 26 is a block diagram illustrating an example of a configuration of a feature quantity data generation device according to a fourth example embodiment.

FIG. 27 is a block diagram illustrating an example of a hardware configuration for executing control or processing according to each example embodiment.

### EXAMPLE EMBODIMENT

Hereinafter, example embodiments of the present invention will be described with reference to the drawings. However, it should be noted that the example embodiments to be described below are limited to be technically preferable in carrying out the present invention, but the scope of the invention is not limited to the following example embodiments. Note that, in all the drawings used to describe the following example embodiments, the same reference signs are given to the same parts unless there is a particular reason. Furthermore, in the following example embodiments, the description of the same configurations and operations may not be repeated.

### First Example Embodiment

First, a gait measurement device according to a first example embodiment will be described with reference to the drawings. The gait measurement device according to the present example embodiment measures sensor data regarding movements of feet measured according to a gait of a user. The gait measurement device according to the present example embodiment generates feature quantity data used in estimating a physical condition of the user, using the measured sensor data.

### (Configuration)

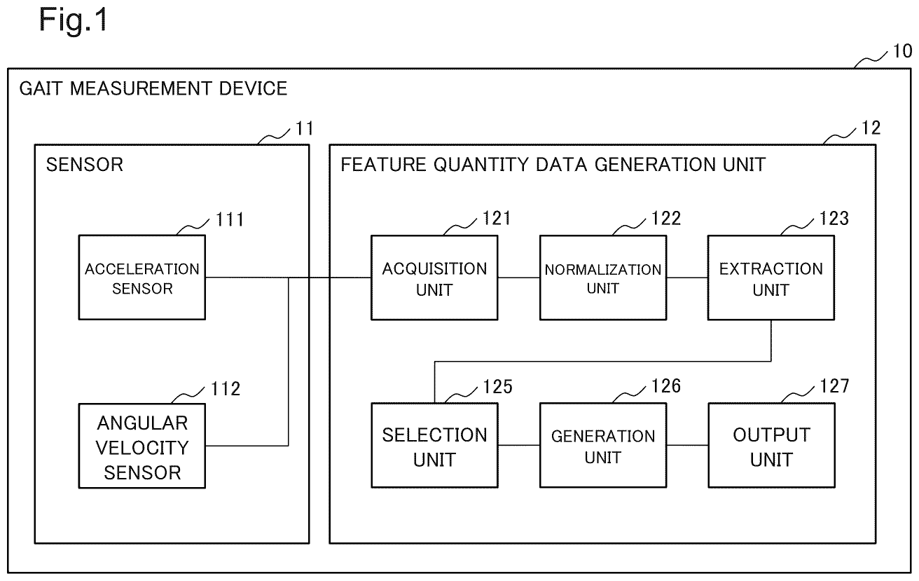

FIG. 1 is a block diagram illustrating an example of a configuration of a gait measurement device 10 according to the present example embodiment. The gait measurement device 10 includes a sensor 11 and a feature quantity data generation unit 12. In the present example embodiment, the gait measurement device 10 in which the sensor 11 and the feature quantity data generation unit 12 are integrated will be described. For example, the gait measurement device 10 is installed on footwear or the like of a subject (a user) who is a target in estimating a physical condition. Hereinafter, the sensor 11 and the feature quantity data generation unit 12 will be individually described.

### [Sensor]

The sensor 11 includes an acceleration sensor 111 and an angular velocity sensor 112. In FIG. 1, the acceleration sensor 111 and the angular velocity sensor 112 are included in the sensor 11 as an example. The sensor 11 may include a sensor other than the acceleration sensor 111 and the angular velocity sensor 112. The sensor other than the acceleration sensor 111 and the angular velocity sensor 112 that can be included in the sensor 11 will not be described.

The acceleration sensor 111 is a sensor that measures accelerations in three axial directions (also referred to as a spatial acceleration). The acceleration sensor 111 measures an acceleration (also referred to as a spatial acceleration) as a physical quantity related to a movement of a foot. The acceleration sensor 111 outputs the measured acceleration to the gait measurement device 10. For example, a piezoelectric type sensor, a piezoresistive type sensor, a capacitance type sensor, or the like can be used as the acceleration sensor 111. The measurement method of the sensor used as the acceleration sensor 111 is not limited as long as the sensor can measure an acceleration.

The angular velocity sensor 112 is a sensor that measures angular velocities around three axes (also referred to as a spatial angular velocity). The angular velocity sensor 112 measures an angular velocity (also referred to as a spatial angular velocity) as a physical quantity related to a movement of a foot. The angular velocity sensor 112 outputs the measured angular velocity to the gait measurement device 10. For example, a vibration type sensor, a capacitance type sensor, or the like can be used as the angular velocity sensor 112. The measurement method of the sensor used as the angular velocity sensor 112 is not limited as long as the sensor can measure an angular velocity.

The sensor 11 is achieved by, for example, an inertial measurement device that measures an acceleration and an angular velocity. An example of the inertial measurement device is an inertial measurement unit (IMU). The IMU includes an acceleration sensor 111 that measures accelerations in three axial directions and an angular velocity sensor 112 that measures angular velocities around three axes. The sensor 11 may be achieved by an inertial measurement device such as a vertical gyro (VG) or an attitude heading (AHRS). Furthermore, the sensor 11 may be achieved by a global positioning system/inertial navigation system (GPS/INS). The sensor 11 may be achieved by a device other than the inertial measurement device as long as the sensor can measure a physical quantity related to a movement of a foot.

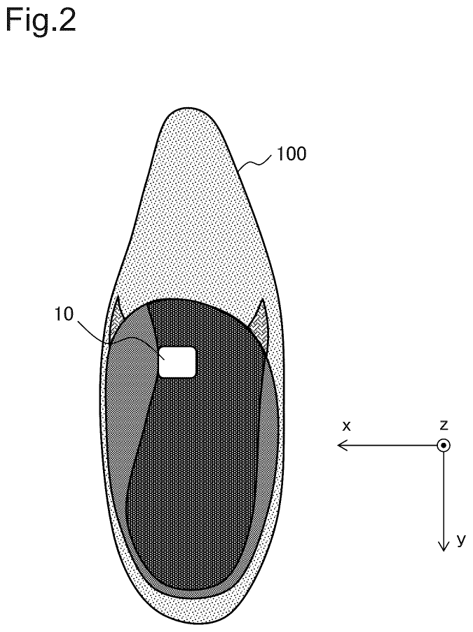

FIG. 2 is a conceptual diagram illustrating an example in which the gait measurement device 10 is arranged in a shoe 100 for a right foot. In the example of FIG. 2, the gait measurement device 10 is installed at a position corresponding to the back side of the arch of foot. For example, the gait measurement device 10 is arranged on an insole inserted into the shoe 100. For example, the gait measurement device 10 may be arranged on the bottom surface of the shoe 100. For example, the gait measurement device 10 may be embedded in the body of the shoe 100. The gait measurement device 10 may be detachable from the shoe 100 or may be undetachable from the shoe 100. The gait measurement device 10 may be installed at a position other than the back side of the arch of foot as long as sensor data related to a movement of a foot can be measured. Furthermore, the gait measurement device 10 may be installed on a sock worn by the user or an accessory such as an anklet worn by the user. Furthermore, the gait measurement device 10 may be directly attached to the foot or may be embedded in the foot. In FIG. 2, the gait measurement device 10 is installed on the shoe 100 for the right foot as an example. The gait measurement devices 10 may be installed on shoes 100 for both feet.

In the example of FIG. 2, a local coordinate system including an x axis in a left-right direction, a y axis in a front-back direction, and a z axis in an up-down direction is set with respect to the gait measurement device 10 (the sensor 11). The left side is positive in the x axis, the back side is positive in the y axis, and the upper side is positive in the z axis. The directions of the axes set in the sensors 11 may be the same for the left and right feet, or may be different for the left and right feet. For example, in a case where the sensors 11 produced with the same specifications are arranged inside the left and right shoes 100, the vertical directions (the Z-axis directions) of the sensors 11 arranged in the left and right shoes 100 are the same. In this case, the three axes of the local coordinate system set in the sensor data derived from the left foot and the three axes of the local coordinate system set in the sensor data derived from the right foot are the same on the left and right sides.

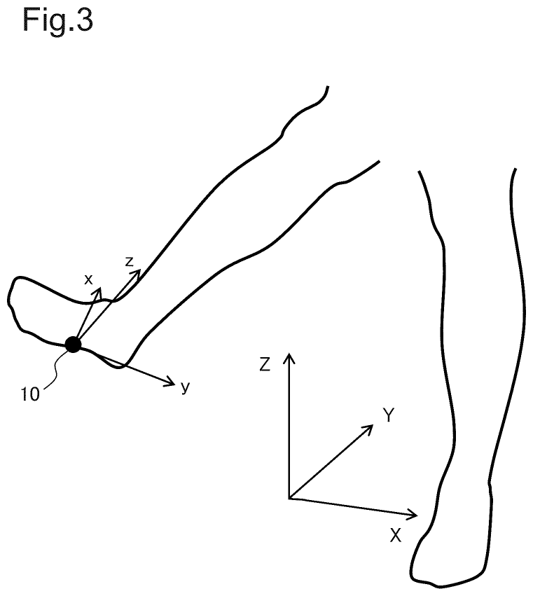

FIG. 3 is a conceptual diagram for explaining a local coordinate system (an x axis, a y axis, and a z axis) set in the gait measurement device 10 (the sensor 11) installed on the back side of the arch of foot and a world coordinate system (an X axis, a Y axis, and a Z axis) set with respect to the ground. In the world coordinate system (the X axis, the Y axis, and the Z axis), in a state where the user facing the moving direction is upright, the lateral direction of the user is set to the X-axis direction (the leftward direction is positive), the back-side direction of the user is set to the Y-axis direction (the backward direction is positive), and the gravity direction is set to the Z-axis direction (the vertically upward direction is positive). Note that, in the example of FIG. 3, although the relationship between the local coordinate system (the x axis, the y axis, and the z axis) and the world coordinate system (the X axis, the Y axis, and the Z axis) is conceptually illustrated, the relationship between the local coordinate system and the world coordinate system, which vary depending on the gait of the user, is not accurately illustrated.

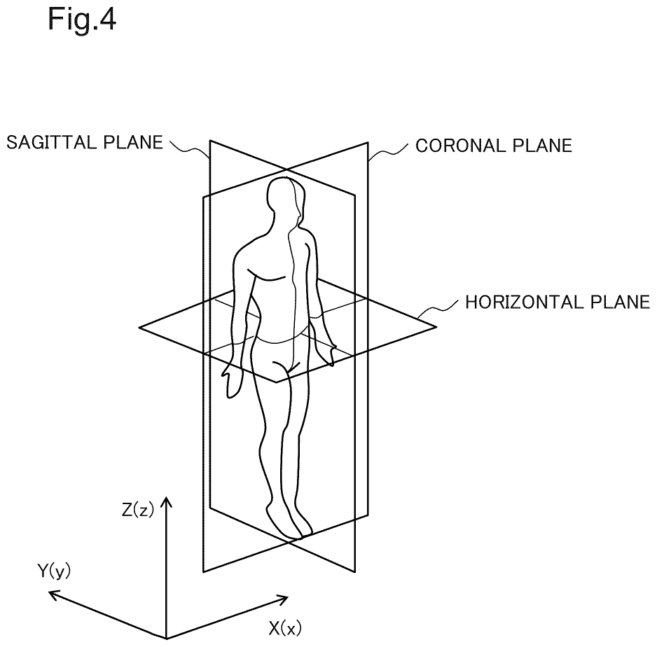

FIG. 4 is a conceptual diagram for explaining planes (also referred to as human body planes) set for a human body. In the present example embodiment, a sagittal plane dividing the body into the left half and the right half, a coronal plane dividing the body into the front half and the rear half, and a horizontal plane dividing the body horizontally are defined. As illustrated in FIG. 4, the world coordinate system and the local coordinate system coincide with each other in a state where the user stands upright with the center line of the foot facing the moving direction. In the present example embodiment, a rotation in the sagittal plane with the x axis as a rotation axis is defined as a roll, a rotation in the coronal plane with the y axis as a rotation axis is defined as a pitch, and a rotation in the horizontal plane with the z axis as a rotation axis is defined as a yaw. In addition, a rotation angle in the sagittal plane with the x axis as a rotation axis is defined as a roll angle, a rotation angle in the coronal plane with the y axis as a rotation axis is defined as a pitch angle, and a rotation angle in the horizontal plane with the z axis as a rotation axis is defined as a yaw angle. In the present example embodiment, when the body is viewed from behind, a counterclockwise rotation in the coronal plane is defined as a positive rotation, and a clockwise rotation in the coronal plane is defined as a negative rotation.

### [Feature Quantity Data Generation Unit]

The feature quantity data generation unit 12 includes an acquisition unit 121, a normalization unit 122, an extraction unit 123, a selection unit 125, a generation unit 126, and an output unit 127. For example, the feature quantity data generation unit 12 is achieved by a microcomputer or a microcontroller that performs overall control and data processing for the gait measurement device 10. For example, the feature quantity data generation unit 12 includes a central processing unit (CPU), a random access memory (RAM), a read only memory (ROM), a flash memory, and the like. The feature quantity data generation unit 12 controls the acceleration sensor 111 and the angular velocity sensor 112 to measure an angular velocity and an acceleration. For example, the feature quantity data generation unit 12 may be implemented on a mobile terminal (not illustrated) carried by the subject (the user).

The acquisition unit 121 acquires accelerations in three axial directions from the acceleration sensor 111. In addition, the acquisition unit 121 acquires angular velocities around three axes from the angular velocity sensor 112. For example, the acquisition unit 121 performs analog-to-digital conversion (AD conversion) on the acquired physical quantities (analog data) such as angular velocities and accelerations. Note that the physical quantities (analog data) measured by the acceleration sensor 111 and the angular velocity sensor 112 may be converted into digital data in the acceleration sensor 111 and the angular velocity sensor 112, respectively. The acquisition unit 121 outputs the converted digital data (also referred to as sensor data) to the normalization unit 122. The acquisition unit 121 may be configured to store the sensor data in a storage unit that is not illustrated. The sensor data includes at least acceleration data converted into digital data and angular velocity data converted into digital data. The acceleration data includes acceleration vectors in three axial directions. The angular velocity data includes angular velocity vectors around three axes. The acceleration data and the angular velocity data are associated with times at which the data are acquired. In addition, the acquisition unit 121 may apply a correction, such as a correction in mounting error or temperature or a correction in linearity, to the acceleration data and the angular velocity data.

The normalization unit 122 acquires the sensor data from the acquisition unit 121. The normalization unit 122 extracts time-series data (also referred to as gait waveform data) for one gait cycle from time-series data on accelerations in three axial directions and angular velocities around three axes included in the sensor data. The normalization unit 122 normalizes a time for the extracted gait waveform data for one gait cycle to a gait cycle of 0 to 100% (percent) (also referred to as first normalization). A timing such as 1% or 10% included in the gait cycle of 0 to 100% is also referred to as a gait phase. Furthermore, the normalization unit 122 normalizes the gait waveform data for one gait cycle subjected to the first normalization so that the stance phase is 60% and the swing phase is 40% (also referred to as second normalization). The stance phase is a period in which at least a partial portion of the back side of the foot is in contact with the ground. The swing phase is a period in which the back side of the foot is separated from the ground. By performing the second normalization on the gait waveform data, it is possible to suppress a deviation of a gait phase in which a feature quantity is extracted from shifting due to the influence of disturbance.

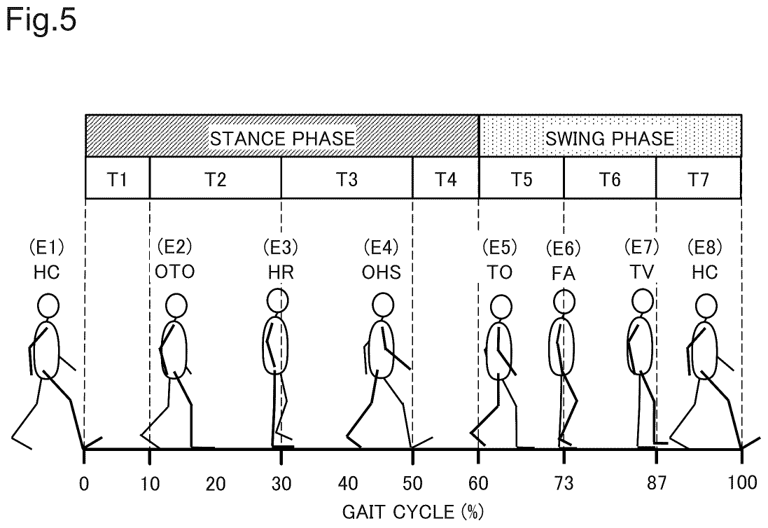

FIG. 5 is a conceptual diagram for explaining one gait cycle based on the right foot. One gait cycle based on the left foot is also similar to that based on the right foot. The horizontal axis of FIG. 5 represents one gait cycle of the right foot, with a time point at which the heel of the right foot lands on the ground as a start point and a time point at which the heel of the right foot next lands on the ground as an end point. In the horizontal axis in FIG. 5, first normalization is performed with one gait cycle as 100%. In addition, in the horizontal axis of FIG. 5, second normalization is performed so that the stance phase is 60% and the swing phase is 40%. The one gait cycle of one foot is roughly divided into a stance phase, in which at least a partial portion of the back side of the foot is in contact with the ground, and a swing phase, in which the back side of the foot is separated from the ground. The stance phase is further subdivided into a load response period T1, a mid-stance period T2, a terminal stance period T3, and a pre-swing period T4. The swing phase is further subdivided into an initial swing period T5, a mid-swing period T6, and a terminal swing period T7. Note that FIG. 5 is an example, and does not limit the periods constituting one gait cycle, the terms for these periods, and the like.

As illustrated in FIG. 5, in a gait, a plurality of events (also referred to as gait events) occur. E1 represents an event in which the heel of the right foot contacts the ground (heel contact (HC)). E2 represents an event in which the toe of the left foot is separated from the ground in a state where the sole of the right foot is in contact with the ground (opposite toe off (OTO)). E3 represents an event in which the heel of the right foot rises in a state where the sole of the right foot is in contact with the ground (heel rise (HR)). E4 is an event in which the heel of the left foot is in contact with the ground (opposite heel strike (OHS)). E5 represents an event in which the toe of the right foot is separated from the ground in a state where the sole of the left foot is in contact with the ground (toe off (TO)). E6 represents an event in which the left foot and the right foot cross each other in a state where the sole of the left foot is in contact with the ground (foot adjacent (FA)). E7 represents an event in which the tibia of the right foot is approximately perpendicular to the ground in a state where the sole of the left foot is in contact with the ground (tibia vertical (TV)). E8 represents an event in which the heel of the right foot contacts the ground (heel contact (HC)). E8 is an end point of the gait cycle starting from E1 and corresponds to a start point of a next gait cycle. Note that FIG. 5 is an example, and does not limit events that occur in a gait or the terms for these events.

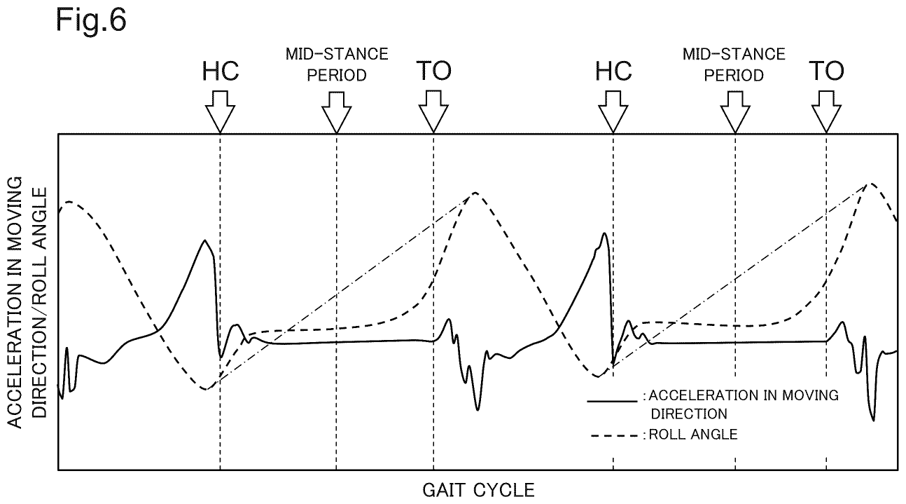

FIG. 6 is a diagram for explaining an example in which a heel contact HC and a toe off TO are detected from time-series data (solid line) on accelerations in the moving direction (accelerations in the Y direction). The timing of the heel contact HC is a timing of a minimum peak immediately after a maximum peak appearing in the time-series data on accelerations in the moving direction (accelerations in the Y direction). The maximum peak serving as a mark of a timing of heel contact HC corresponds to a largest peak of gait waveform data for one gait cycle. A section between consecutive heel contacts HC is one gait cycle. The timing of toe off TO is a rising timing of a maximum peak appearing after the period of the stance phase in which no variation occur in time-series data on accelerations in the moving direction (accelerations in the Y direction). FIG. 6 also illustrates time-series data (broken line) on roll angles (angular velocities around the X axis). A midpoint timing between a timing of a smallest roll angle and a timing of a largest roll angle corresponds to the mid-stance period. For example, parameters such as gait speed, stride, circumduction, internal/external rotation, and plantarflexion/dorsiflexion (also referred to as gait parameters) can be obtained based on the mid-stance period.

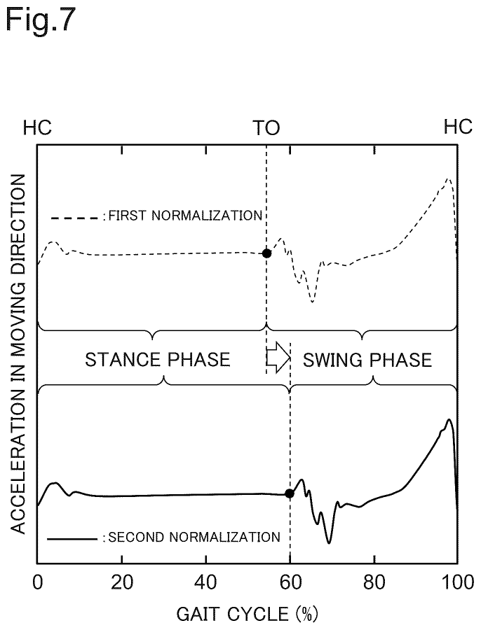

FIG. 7 is a diagram for explaining an example of gait waveform data normalized by the normalization unit 122. The normalization unit 122 detects a heel contact HC and a toe off TO from time-series data on accelerations in the moving direction (accelerations in the Y direction). The normalization unit 122 extracts a section between consecutive heel contacts HC as gait waveform data for one gait cycle. The normalization unit 122 converts the horizontal axis (time axis) of the gait waveform data for one gait cycle into a gait cycle of 0 to 100% by performing first normalization. In FIG. 7, gait waveform data after first normalization is indicated by a broken line. In the gait waveform data (broken line) after the first normalization, the timing of toe off TO deviates from 60%.

In the example of FIG. 7, the normalization unit 122 normalizes a section from the heel contact HC at which the gait phase is 0% to the toe off TO after the heel contact HC to 0 to 60%. In addition, the normalization unit 122 normalizes a section from the toe off TO to heel contact HC at which the gait phase is 100% after the toe off TO to 60 to 100%. As a result, the gait waveform data for one gait cycle is normalized to a section (a stance phase) in which the gait cycle is 0 to 60% and a section (a swing phase) in which the gait cycle is 60 to 100%. In FIG. 7, gait waveform data after second normalization is indicated by a solid line. In the gait waveform data (solid line) after the second normalization, the timing of toe off TO coincides with 60%.

In FIGS. 6 and 7, the examples in which the gait waveform data for one gait cycle is extracted/normalized based on the accelerations in the moving direction (accelerations in the Y direction) are illustrated. With respect to accelerations other than the accelerations in the moving direction (accelerations in the Y direction) and angular velocities, the normalization unit 122 extracts/normalizes gait waveform data for one gait cycle in line with the gait cycle for the accelerations in the moving direction (accelerations in the Y direction). Furthermore, the normalization unit 122 may generate time-series data on angles around three axes by integrating the time-series data on angular velocities around three axes. In this case, the normalization unit 122 also extracts/normalizes gait waveform data for one gait cycle, with respect to the angles around the three axes, in line with the gait cycle for the accelerations in the moving direction (accelerations in the Y direction).

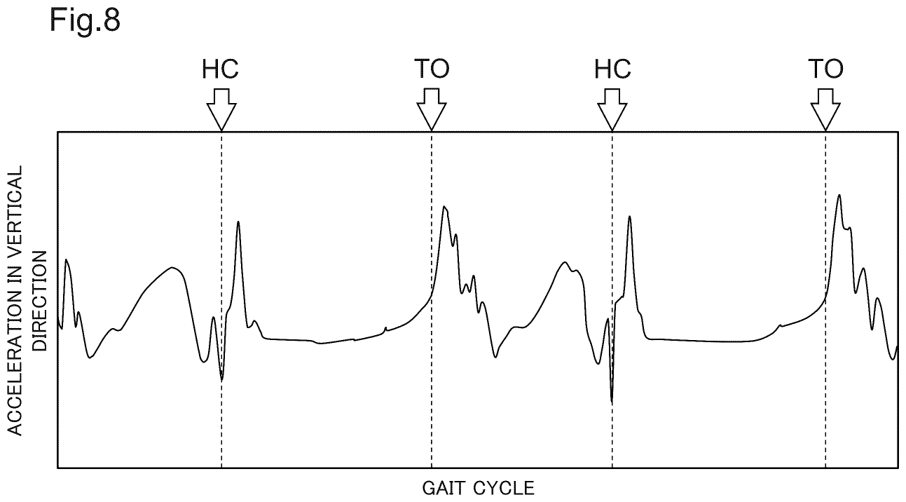

The normalization unit 122 may extract/normalize gait waveform data for one gait cycle based on accelerations other than the accelerations in the moving direction (accelerations in the Y direction) and angular velocities. FIG. 8 is a diagram for explaining an example in which a heel contact HC and a toe off TO are detected from time-series data on accelerations in the vertical direction (accelerations in the Z direction). The timing of heel contact HC is a timing of a steep minimum peak appearing in the time-series data on accelerations in the vertical direction (accelerations in the Z direction). At the timing of the steep minimum peak, the value of the acceleration in the vertical direction (the acceleration in the Z direction) is substantially zero. The minimum peak serving as a mark of a timing of heel contact HC corresponds to a smallest peak of gait waveform data for one gait cycle. A section between consecutive heel contacts HC is one gait cycle. The timing of toe off TO is a timing of an inflection point in the middle in which a variation gradually increases after the time-series data on accelerations in the vertical direction (accelerations in the Z direction) passes through a section in which the variation is small after a maximum peak immediately after the heel contact HC.

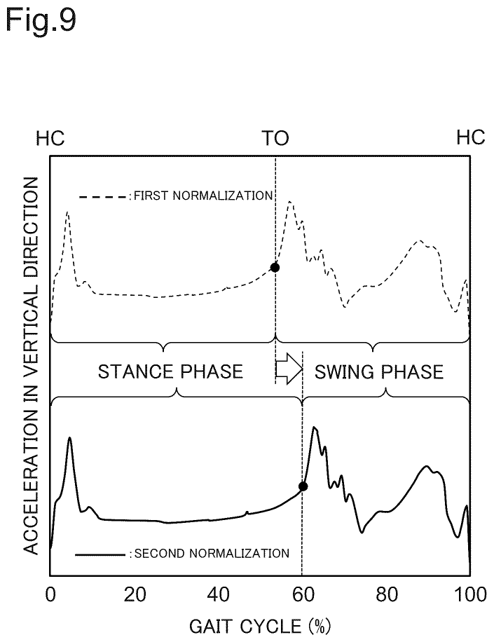

FIG. 9 is a diagram for explaining an example of gait waveform data normalized by the normalization unit 122. For example, the normalization unit 122 detects a heel contact HC and a toe off TO from time-series data on accelerations in the vertical direction (accelerations in the Z direction). The normalization unit 122 extracts a section between consecutive heel contacts HC as gait waveform data for one gait cycle. The normalization unit 122 converts the horizontal axis (time axis) of the gait waveform data for one gait cycle into a gait cycle of 0 to 100% by performing first normalization. In FIG. 7, gait waveform data after first normalization is indicated by a broken line. In the gait waveform data (broken line) after the first normalization, the timing of toe off TO deviates from 60%.

In the example of FIG. 9, the normalization unit 122 normalizes a section from the heel contact HC at which the gait phase is 0% to the toe off TO after the heel contact HC to 0 to 60%. In addition, the normalization unit 122 normalizes a section from the toe off TO to heel contact HC at which the gait phase is 100% after the toe off TO to 60 to 100%. As a result, the gait waveform data for one gait cycle is normalized to a section (a stance phase) in which the gait cycle is 0 to 60% and a section (a swing phase) in which the gait cycle is 60 to 100%. In FIG. 7, gait waveform data after second normalization is indicated by a solid line. In the gait waveform data (solid line) after the second normalization, the timing of toe off TO coincides with 60%.

In FIGS. 8 and 9, the examples in which the gait waveform data for one gait cycle is extracted/normalized based on the accelerations in the vertical direction (accelerations in the Z direction) are illustrated. With respect to accelerations other than the accelerations in the vertical direction (accelerations in the Z direction) and angular velocities, the normalization unit 122 extracts/normalizes gait waveform data for one gait cycle in line with the gait cycle for the accelerations in the vertical direction (accelerations in the Z direction). Furthermore, the normalization unit 122 may generate time-series data on angles around three axes by integrating the time-series data on angular velocities around three axes. In this case, the normalization unit 122 also extracts/normalizes gait waveform data for one gait cycle, with respect to the angles around the three axes, in line with the gait cycle for the accelerations in the vertical direction (accelerations in the Z direction). Furthermore, the normalization unit 122 may extract/normalize gait waveform data for one gait cycle based on both accelerations in the moving direction (accelerations in the Y direction) and accelerations in the vertical direction (accelerations in the Z direction). Furthermore, the normalization unit 122 may extract/normalize gait waveform data for one gait cycle based on accelerations other than the accelerations in the moving direction (the accelerations in the Y direction) and the accelerations in the vertical direction (the accelerations in the Z direction), angular velocities, angles, and the like.

The extraction unit 123 acquires the gait waveform data for one gait cycle normalized by the normalization unit 122. The extraction unit 123 extracts feature quantities according to the physical condition of the estimation target from the gait waveform data for one gait cycle. The extraction unit 123 extracts feature quantities for each gait phase cluster from gait phase clusters each being obtained by integrating temporally consecutive gait phases based on a preset condition. For example, the extraction unit 123 extracts feature quantities used in estimating a first metatarsophalangeal angle (FMTPA) of the subject. For example, the extraction unit 123 extracts feature quantities used in estimating a center of pressure excursion index (CPEI). Note that the physical condition of the estimation target is not limited to the first metatarsophalangeal angle (FMTPA) or the center of pressure excursion index (CPEI) as long as the physical condition can be estimated based on the sensor data regarding movements of feet.

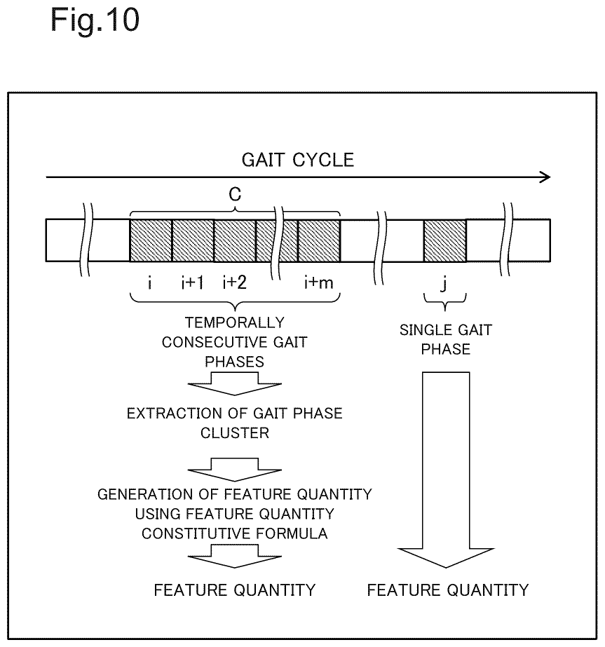

FIG. 10 is a conceptual diagram for explaining extraction of feature quantities for estimating a physical condition from gait waveform data for one gait cycle. For example, the extraction unit 123 extracts temporally consecutive gait phases i to i+m as a gait phase cluster C (i and m are natural numbers). The gait phase cluster C includes m gait phases (components). That is, the number of gait phases (components) (also referred to as the number of components) constituting the gait phase cluster C is m. Although the gait phase has an integer value as an example in FIG. 10, the value of the gait phase may be subdivided into a value having a decimal place. In a case where the value of the gait phase is subdivided into a value having a decimal place, the number of components of the gait phase cluster C is a number corresponding to the number of data points in the section of the gait phase cluster. The extraction unit 123 extracts a feature quantity from each of the gait phases i to i+m. In a case where the gait phase cluster C includes a single gait phase j, the extraction unit 123 extracts a feature quantity from the single gait phase j (j is a natural number).

The selection unit 125 selects a feature quantity having a small variation among the feature quantities for estimating a physical condition extracted by the extraction unit 123. The smaller the number of components of the gait phase cluster, the larger the error caused by the forward or backward deviations of the gait phases, increasing the variation. Therefore, for example, the selection unit 125 selects a feature quantity having a small variation according to the number of components of the gait phase cluster. For example, the selection unit 125 selects a feature quantity using a preset threshold (also referred to as a selection threshold) with respect to the number of components of the gait phase cluster as a reference. For example, when the selection threshold is 5, the selection unit 125 selects a gait phase cluster having five or more components. In other words, when the selection threshold is 5, the selection unit 125 excludes a gait phase cluster having four or less components. When the number of components of the gait phase cluster is five or more, feature quantities of gait phases constituting the same gait phase cluster are averaged, so that an influence of a gait phase having a large variation in the feature quantity can be alleviated. The selection threshold set with respect to the number of components of the gait phase cluster is not limited to five, and any value can be set.

Here, the selection of the feature quantity by the selection unit 125 will be described by exemplifying hallux valgus. A degree of hallux valgus can be evaluated by a first metatarsophalangeal angle FMTPA. The first metatarsophalangeal angle FMTPA is a metatarsophalangeal angle of a first toe (thumb). In the present example embodiment, in a case where the first metatarsophalangeal angle FMTPA exceeds 25 degrees, the case is classified as having hallux valgus. In a case where the first metatarsophalangeal angle FMTPA is 15 degrees or more and 25 degrees or less, the case is classified as having a tendency toward hallux valgus. In a case where the first metatarsophalangeal angle FMTPA is less than 15 degrees, the case is classified as being normal.

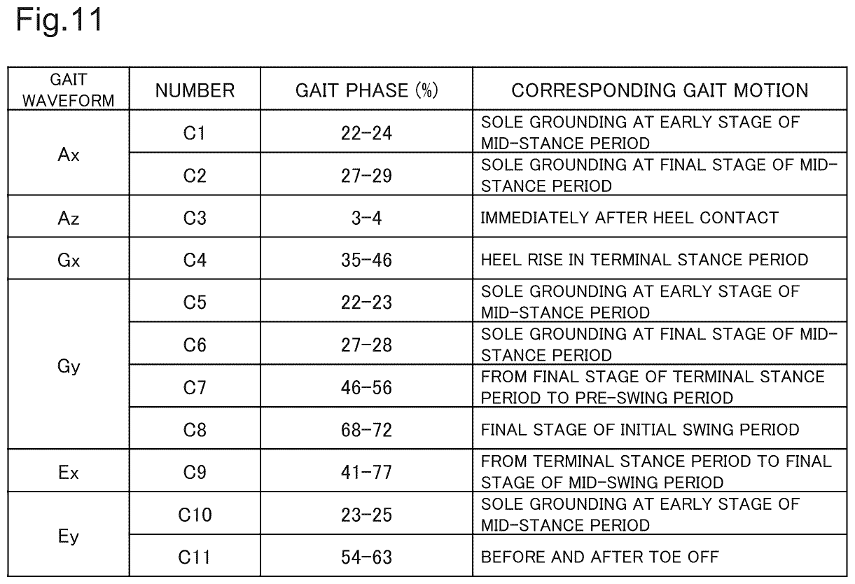

FIG. 11 is a correspondence table summarizing feature quantities used in estimating a first metatarsophalangeal angle FMTPA. In the correspondence table of FIG. 11, gait waveform data from which a feature quantity is extracted, a gait phase cluster number, a gait phase (%) in which the gait phase cluster is extracted, the number of components, and a corresponding gait motion.

The gait waveform data Ax is gait waveform data for one gait cycle related to time-series data on accelerations in the lateral direction (accelerations in the X direction). The gait waveform data Ax includes two gait phase clusters. The gait phase cluster C1 is a section of a gait phase of 22 to 24%. The number of components of the gait phase cluster C1 is three. The gait motion corresponding to the section of the gait phase of 22 to 24% is sole grounding at the early stage of the mid-stance period. The gait phase cluster C2 is a section of a gait phase of 27 to 29%. The number of components of the gait phase cluster C1 is three. The gait motion corresponding to the gait phase of 27 to 29% is sole grounding at the final stage of the mid-stance period.

The gait waveform data Az is gait waveform data for one gait cycle related to time-series data on accelerations in the vertical direction (accelerations in the Z direction). The gait waveform data Az includes one gait phase cluster. The gait phase cluster C3 is a section of a gait phase of 3 to 4%. The number of components of the gait phase cluster C3 is two. The gait motion corresponding to the section of the gait phase of 3 to 4% is immediately after the heel contact.

The gait waveform data Gx is gait waveform data for one gait cycle related to time-series data on angular velocities (roll angular velocities) around the X axis. The gait waveform data Gx includes one gait phase cluster. The gait phase cluster C4 is a section of a gait phase of 35 to 46%. The number of components of the gait phase cluster C4 is 12. The gait motion corresponding to the section of the gait phase of 35 to 46% is the heel rise in the terminal stance period.

The gait waveform data Gy is gait waveform data for one gait cycle related to time-series data on angular velocities (pitch angular velocities) around the Y axis. The gait waveform data Gy includes four gait phase clusters. The gait phase cluster C5 is a section of a gait phase of 22 to 23%. The number of components of the gait phase cluster C5 is two. The gait motion corresponding to the section of the gait phase of 22 to 23% is sole grounding at the early stage of the mid-stance period. The gait phase cluster C6 is a section of a gait phase of 27 to 28%. The number of components of the gait phase cluster C6 is two. The gait motion corresponding to the section of the gait phase of 27 to 28% is sole grounding at the final stage of the mid-stance period. The gait phase cluster C7 is a section of a gait phase of 46 to 56%. The number of components of the gait phase cluster C7 is 11. The gait motion corresponding to the section of the gait phase of 46 to 56% is from the final stage of the terminal stance period to the pre-swing period. The gait phase cluster C8 is a section of a gait phase of 68 to 72%. The number of components of the gait phase cluster C8 is five. The gait motion corresponding to the section of the gait phase of 68 to 72% is the final stage of the initial swing period.

The gait waveform data Ex is gait waveform data for one gait cycle related to time-series data on attitude angles (roll angles) around the X axis. The attitude angle (roll angle) around the X axis is obtained by integrating an angular velocity (roll angular velocity) around the X axis. The gait waveform data Ex includes one gait phase cluster. The gait phase cluster C9 is a section of a gait phase of 41 to 77%. The number of components of the gait phase cluster C9 is 12. The gait motion corresponding to the section of the gait phase of 41 to 77% is from the terminal stance period to the final stage of the mid-swing period.

The gait waveform data Ey is gait waveform data for one gait cycle related to time-series data on attitude angles (pitch angles) around the Y axis. The attitude angle (pitch angle) around the Y axis is obtained by integrating an angular velocity (pitch angular velocity) around the Y axis. The gait waveform data Ey includes two gait phase clusters. The gait phase cluster C10 is a section of a gait phase of 23 to 25%. The number of components of the gait phase cluster C10 is 2. The gait motion corresponding to the section of the gait phase of 23 to 25% is sole grounding at the early stage of the mid-stance period. The gait phase cluster C11 is a section of a gait phase of 54 to 63%. The number of components of the gait phase cluster C11 is 10. The gait motion corresponding to the section of the gait phase of 54 to 63% is before and after the toe off.

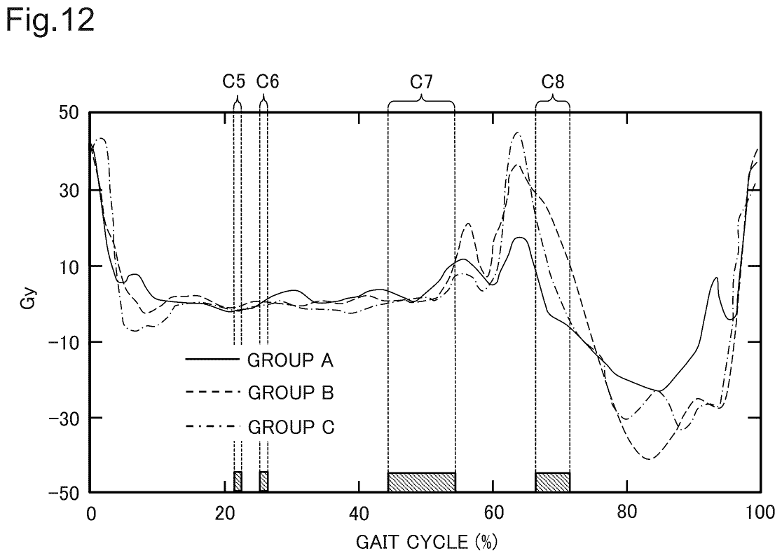

FIG. 12 is an example of gait waveform data from which feature quantities used in estimating a first metatarsophalangeal angle FMTPA are extracted. FIG. 12 is gait waveform data Gy for one gait cycle related to time-series data on angular velocities (pitch angular velocities) around the Y axis. FIG. 12 relates to verification performed on 50 subjects. The verification of FIG. 12 was performed under the condition that the subjects wearing shoes in which the measurement devices are installed walk at a comfortable speed without specifying a gait speed or the like. The measurements were carried out by 50 subjects under the same conditions in a sequence of four round trips over a distance of 8 meters. The 50 subjects were classified into group A having a first metatarsophalangeal angle FMTPA of more than 25 degrees, group B having a first metatarsophalangeal angle FMTPA of 15 degrees or more and 25 degrees or less, and group C having a first metatarsophalangeal angle FMTPA of less than 15 degrees. In FIG. 12, a waveform for group A is indicated by a solid line, a waveform for group B is indicated by a broken line, and a waveform for group C is indicated by a one-dot chain line. In the sequence of four round trips over a distance of 8 meters, sensor data for about 50 steps was acquired. The sensor data acquired from each subject is averaged according to the number of steps.

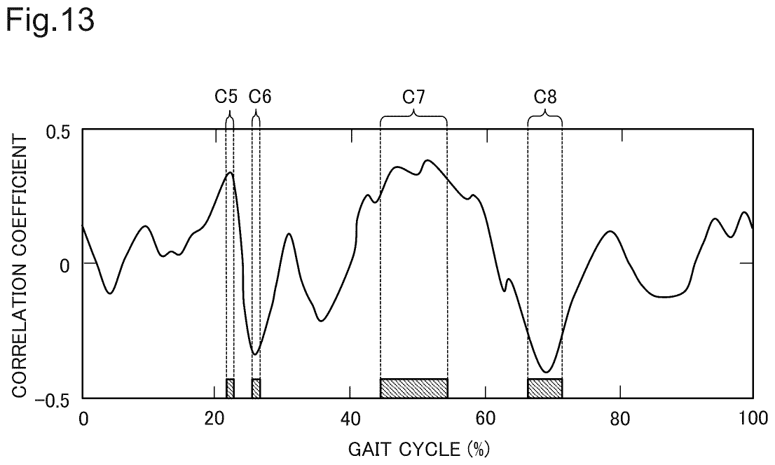

The graph of FIG. 13 is a graph of a correlation coefficient between a value (first metatarsophalangeal angle FMTPA) related to a physical condition of an estimation target and a feature quantity. A gait phase (%) in which the maximum/minimum correlation coefficient between the first metatarsophalangeal angle FMTPA and the feature quantity is remarkable constitutes a gait phase cluster. In the example of FIG. 13, the maximum/minimum correlation coefficients are remarkable for the gait phase clusters C5 to C8.

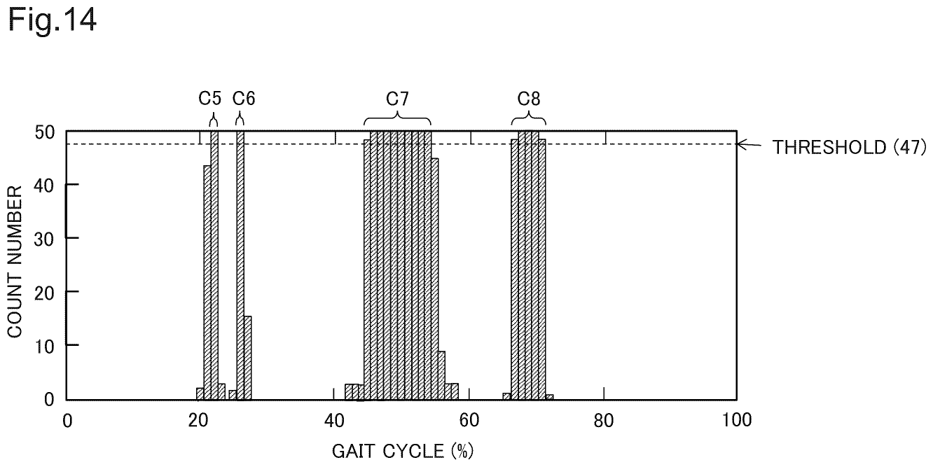

The graph of FIG. 14 shows the number of times (also referred to as a count number) it is determined, by leave-one-subject-out correlation analysis, that correlation between a value (first metatarsophalangeal angle FMTPA) related to a physical condition of an estimation target and a feature quantity is significant. In the leave-one-subject-out correlation analysis, in order to verify whether a value output by the estimation model follows an essential distribution of data while removing individual difference factors, correlation analysis is performed by sequentially excluding one by one. In this example of verification, analysis on correlation using feature quantity data for 49 subjects excluding feature quantity data for 1 subject from feature quantity data for 50 subjects was repeated 50 times. In this verification, the threshold of the count number is set to 47, and a feature quantity having a count number of 47 or more is extracted. A feature quantity having a count number of less than 47 was considered to have essentially no influence of hallux valgus. A feature quantity having a count number of less than 47 causes a low correlation, and thus is not extracted as a feature quantity of a gait phase cluster. The threshold of the count number may be set according to the purpose.

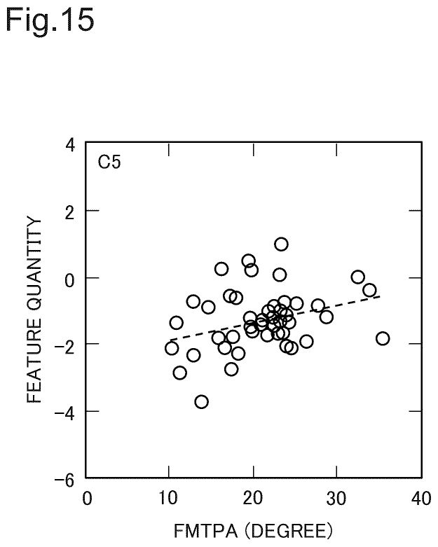

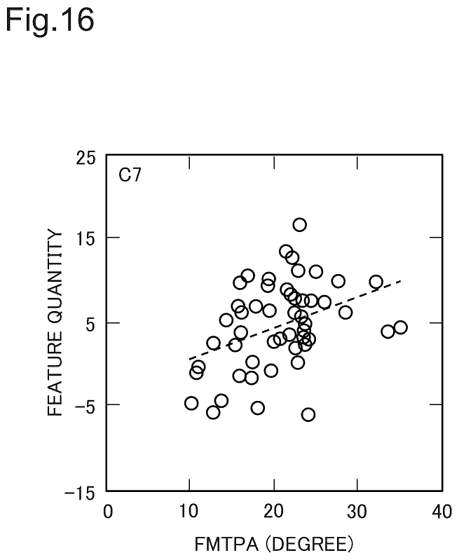

Next, a variation of a first metatarsophalangeal angle FMTPA depending on the number of components of a gait phase cluster will be described by comparing the gait phase cluster C5 and the gait phase cluster C7. FIG. 15 is a graph illustrating a relationship between a value of a first metatarsophalangeal angle FMTPA and a feature quantity regarding the gait phase cluster C5. FIG. 16 is a graph illustrating a relationship between a value of a first metatarsophalangeal angle FMTPA and a feature quantity regarding the gait phase cluster C7. The feature quantity in FIGS. 15 and 16 is an integrated average value of signal intensities for each gait phase cluster. In FIGS. 15 and 16, a regression line (broken line) is shown by fitting the relationship between the value of the first metatarsophalangeal angle FMTPA and the feature quantity to a linear function. As compared with the gait phase cluster C7 (FIG. 16) having a large number of components, in the gait phase cluster C5 (FIG. 15) having a small number of components, the first metatarsophalangeal angle FMTPA greatly varies while the feature quantity slightly varies. In other words, as compared with the gait phase cluster C5 (FIG. 15) having a small number of components, in the gait phase cluster C7 (FIG. 16) having a large number of components, the first metatarsophalangeal angle FMTPA does not change significantly when the feature quantity varies slightly. That is, the smaller the number of components of the gait phase cluster, the more sharply the estimated value of the physical condition of the estimation target changes with respect to the change in feature quantity. Therefore, the selection unit 125 selects a feature quantity of a gait phase cluster having a large number of components, of which an estimated value hardly changes with respect to the change in feature quantity. In other words, the selection unit 125 excludes a feature quantity of a gait phase cluster having a small number of components, of which an estimated value is likely to change with respect to the change in feature quantity is small. For example, the selection unit 125 selects a gait phase cluster depending on whether the number of components is larger or smaller than the preset selection threshold. The selection unit 125 selects a gait phase cluster in which the number of components is equal to or larger than the selection threshold. That is, the selection unit 125 excludes a gait phase cluster in which the number of components is smaller than the selection threshold.

The selection unit 125 may determine a gait phase cluster to be excluded depending on a value of a feature quantity. For example, a threshold (also referred to as a variation threshold) of a feature quantity is set in advance for each gait phase cluster. The variation threshold is set to a value to which an estimated value of a physical condition of an estimation target does not indicate an abnormal value. When a value of a feature quantity related to a gait phase cluster exceeds the variation threshold, the feature quantity related to the gait phase cluster may be overestimated, and an estimated value of a physical condition of an estimation target may indicate an abnormal value. In a case where a physical condition of an estimation target is estimated by a multiple regression prediction method using feature quantities extracted from a plurality of gait phase clusters, weighting is performed for each feature quantity by multiplying a coefficient for each of the plurality of feature quantities. A value of a feature quantity extracted from a gait phase cluster having a small number of components is smaller than a value of a feature quantity extracted from a gait phase cluster having a large number of components. Therefore, a large coefficient is applied to a value of a feature quantity extracted from a gait phase cluster having a small number of components as compared with a value of a feature quantity extracted from a gait phase cluster having a large number of components. Accordingly, a variation in feature quantity extracted from a gait phase cluster having a small number of components may have a great influence on an estimated value of a physical condition. If a feature quantity extracted from a gait phase cluster having a small number of components is excluded, an influence of a variation in feature quantity on an estimation value is reduced.

The selection unit 125 may determine a gait phase cluster to be excluded depending on the number of digits of the value of the feature quantity. For example, the selection unit 125 excludes a feature quantity of which a value has varied by two or more digits as compared with the feature quantities of the other gait phase clusters. For example, the selection unit 125 excludes a feature quantity of which a value has varied by two or more digits.

When the value of the feature quantity exceeds the variation threshold, the selection unit 125 may scan feature quantities in gait phases before and after the gait phase in which the feature quantity is extracted. For example, the selection unit 125 scans feature quantities in gait phases within 5 points before and after the gait phase in which the feature quantity exceeding the variation threshold is extracted. In a case where the number of components of a gait phase cluster is small, a gait phase in which a feature related to a physical condition appears may deviate forward or backward. In such a case, the feature of the physical condition may be included before or after the gait phase in which the feature related to the physical condition is assumed to appear. Therefore, if gait phases within about five points before and after the gait phase in which the feature quantity exceeding the variation threshold is extracted are scanned, there is a possibility that a feature related to a physical condition can be extracted. For example, when a feature quantity lower than the variation threshold is extracted before or after the gait phase in which the feature quantity exceeding the variation threshold is extracted, the selection unit 125 selects the feature quantity of the gait phase.

For example, when the subject (user) walks obliquely, a variation in acceleration in the vertical direction (acceleration in the Z direction) increases even in the period of the stance phase. Normally, in the stance period, since the foot is in contact with the ground, an acceleration in the moving direction (acceleration in the Y direction) and an acceleration in the lateral direction (acceleration in the X direction) are substantially zero. However, when the subject (user) walks obliquely, an acceleration in the oblique direction is detected by the sensor 11, and an acceleration in the moving direction (acceleration in the Y direction) and an acceleration in the lateral direction (acceleration in the X direction) are detected. If a feature quantity extracted from such sensor data is used, a physical condition may be erroneously determined. In addition, a steep measurement value measured due to a factor such as noise also leads to an erroneous determination of a physical condition. By removing the feature quantity exceeding the variation threshold, it is possible to suppress an erroneous determination of a physical condition due to a factor such as oblique walking or noise.

The generation unit 126 applies a feature quantity constitutive formula to the feature quantity (first feature quantity) extracted from each of the gait phases constituting the gait phase cluster to generate a feature quantity (second feature quantity) of the gait phase cluster. The feature quantity constitutive formula is a preset calculation formula for generating a feature quantity of a gait phase cluster. For example, the feature quantity constitutive formula is a calculation formula related to the four fundamental arithmetic operations. For example, the second feature quantity calculated using the feature quantity constitutive formula is an integral average value, an arithmetic average value, a slope, a variation, or the like between the first feature quantities of the respective gait phases included in the gait phase cluster. For example, the generation unit 126 applies a calculation formula for calculating a slope or a variation between the first feature quantities extracted from the respective gait phases constituting the gait phase cluster as the feature quantity constitutive formula. For example, in a case where the gait phase cluster is constituted by a single gait phase, it is not possible to calculate a slope or a variation, and thus, a feature quantity constitutive formula for calculating an integral average value, an arithmetic average value, or the like may be used. The generation unit 126 outputs feature quantity data including the generated feature quantity for each gait phase cluster.

The output unit 127 outputs the feature quantity data generated by the generation unit 126. The output unit 127 outputs the generated feature quantity data of the gait phase cluster to an external system or the like by which the feature quantity data is to be used.

The use of the feature quantity data of the gait phase cluster output from the gait measurement device 10 is not particularly limited. For example, the gait measurement device 10 is connected to an external system or the like constructed in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile phone. For example, the output unit 127 is connected to the mobile terminal via a wire such as a cable. For example, the output unit 127 is connected to the mobile terminal through wireless communication. For example, the gait measurement device 10 is connected to the mobile terminal through a wireless communication function (not illustrated) conforming to a standard such as Bluetooth (registered trademark) or WiFi (registered trademark). Note that the communication function of the gait measurement device 10 may conform to a standard other than Bluetooth (registered trademark) or WiFi (registered trademark). The feature quantity data of the gait phase cluster may be used by an application installed in the mobile terminal. In this case, the mobile terminal processes the feature quantity data of the gait phase cluster by application software or the like installed in the mobile terminal.

### (Operation)

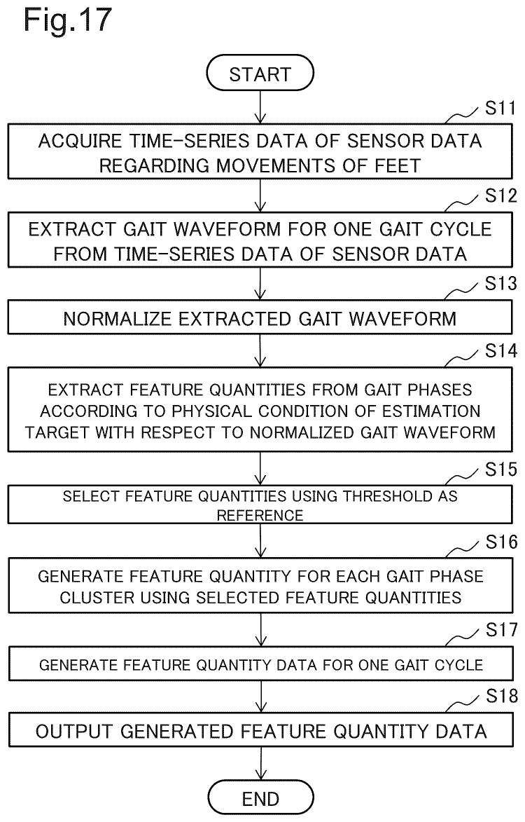

Next, an operation of the gait measurement device 10 will be described with reference to the drawings. Here, an operation of the feature quantity data generation unit 12 included in the gait measurement device 10 will be described. FIG. 17 is a flowchart for explaining the operation of the feature quantity data generation unit 12. In the description based on the flowchart of FIG. 17, the feature quantity data generation unit 12 will be described as an operation subject.

In FIG. 17, first, the feature quantity data generation unit 12 acquires time-series data of sensor data regarding movements of feet (step S11).

Next, the feature quantity data generation unit 12 extracts gait waveform data for one gait cycle from the time-series data of the sensor data (step S12). The feature quantity data generation unit 12 detects heel contact and toe off from the time-series data of the sensor data. The feature quantity data generation unit 12 extracts time-series data for a section between consecutive heel contacts as the gait waveform data for one gait cycle.

Next, the feature quantity data generation unit 12 normalizes the extracted gait waveform data for one gait cycle (step S13). The feature quantity data generation unit 12 normalizes the gait waveform data for one gait cycle to a gait cycle of 0 to 100% (first normalization). Furthermore, the feature quantity data generation unit 12 normalizes a ratio of a stance phase to a swing phase with respect to the gait waveform data for one gait cycle subjected to the first normalization to 60:40 (second normalization).

Next, the feature quantity data generation unit 12 extracts feature quantities from gait phases according to a physical condition of an estimation target with respect to the normalized gait waveform (step S14). For example, the feature quantity data generation unit 12 extracts feature quantities according to a physical condition such as the first metatarsophalangeal angle FMTPA or a center of pressure excursion index CPEI.

Next, the feature quantity data generation unit 12 selects feature quantities using a threshold as a reference, the threshold being set in advance regarding the number of components of a gait phase cluster (step S15). For example, the feature quantity data generation unit 12 selects a gait phase cluster in which the number of components is equal to or larger than the selection threshold. For example, the feature quantity data generation unit 12 excludes a gait phase cluster in which the number of components is smaller than the selection threshold. For example, the feature quantity data generation unit 12 removes a feature quantity exceeding a variation threshold.

Next, the feature quantity data generation unit 12 generates a feature quantity for each gait phase cluster using the selected feature quantities (step S16).

Next, the feature quantity data generation unit 12 generates feature quantity data for one gait cycle by integrating the feature quantities for the respective gait phase clusters (step S17).

Next, the feature quantity data generation unit 12 outputs the generated feature quantity data (step S18).

As described above, the gait measurement device according to the present example embodiment includes a sensor and a feature quantity data generation unit. The sensor includes an acceleration sensor and an angular velocity sensor. The sensor measures spatial accelerations using the acceleration sensor. The sensor measures spatial angular velocities using the angular velocity sensor. The sensor generates sensor data regarding movements of feet using the measured spatial accelerations and spatial angular velocities. The sensor transmits the generated sensor data to the feature quantity data generation device.

The feature quantity data generation device includes an acquisition unit, a normalization unit, an extraction unit, a selection unit, a generation unit, and an output unit. The acquisition unit acquires time-series data of sensor data regarding movements of feet. The normalization unit extracts gait waveform data for one gait cycle from the time-series data of the sensor data, and normalizes the extracted gait waveform data. The extraction unit extracts, from the normalized gait waveform data, feature quantities related to a physical condition of an estimation target from gait phase clusters each constituted by one or more temporally consecutive gait phases. The selection unit selects feature quantities to be used in estimating the physical condition from the extracted feature quantities for the respective gait phase clusters using a preset threshold as a reference. The generation unit generates feature quantity data including the selected feature quantities. The output unit outputs the generated feature quantity data.

The gait measurement device according to the present example embodiment normalizes a gait waveform for one gait cycle, and selects feature quantities to be used in estimating a physical condition using a preset threshold as a reference. Therefore, according to the present example embodiment, it is possible to generate feature quantity data with which a physical condition can be estimated with high accuracy.

In one aspect of the present example embodiment, when a value of any of the extracted feature quantities of the gait phase clusters exceeds a variation threshold set in advance for each of the feature quantities of the gait phase clusters, the selection unit removes the feature quantity exceeding the variation threshold. According to the present aspect, in a case where an abnormal value of the feature quantity is detected, it is possible to eliminate an abnormality that can be contained in the feature quantity data to be used in estimating the physical condition by deleting the feature quantity indicating the abnormal value.

In one aspect of the present example embodiment, when the value of any of the extracted feature quantities of the gait phase clusters exceeds the variation threshold, the selection unit scans feature quantities of gait phases before and after the gait phase constituting the gait phase cluster, and selects a feature quantity lower than the variation threshold. According to the present aspect, when an abnormal value of the feature quantity is detected, a normal feature quantity can be extracted from a gait phase before or after the gait phase in which the feature quantity indicating the abnormal value is detected.

In one aspect of the present example embodiment, the selection unit selects feature quantities of gait phase clusters in each of which the number of components that are gait phases constituting the gait phase cluster exceeds a selection threshold. According to the present aspect, by selecting a gait phase cluster having a large number of components, which has high resistant to noise or the like, it is possible to generate feature quantity data with which a physical condition can be estimated with high accuracy. In other words, according to the present aspect, by removing a gait phase cluster having a small number of components, which has low resistance to noise or the like, it is possible to generate feature quantity data with which a physical condition can be estimated with high accuracy.

In one aspect of the present example embodiment, the normalization unit detects a timing of a heel contact and a timing of a toe off from the time-series data of the sensor data. The normalization unit extracts a section between consecutive heel contacts as the gait waveform data for one gait cycle. The normalization unit executes first normalization in which the gait cycle of the gait waveform data is set in such a way that the preceding heel contact is 0% and the following heel contact is 100%. The normalization unit executes second normalization in which a section between the preceding heel contact and the toe off is 60% and a section between the toe off and the following heel contact is 40%. According to the present aspect, it is possible to suppress a shift of a timing of a gait event such as a heel contact or a toe off detected from the gait waveform data for one gait cycle. Therefore, according to the present aspect, it is possible to generate feature quantity data with which a physical condition can be estimated with high accuracy.

### Second Example Embodiment

Next, a physical condition estimation system according to a second example embodiment will be described with reference to the drawings. The physical condition estimation system according to the present example embodiment estimates a physical condition of a user based on sensor data regarding movements of feet measured according to a gait of the user.

### (Configuration)

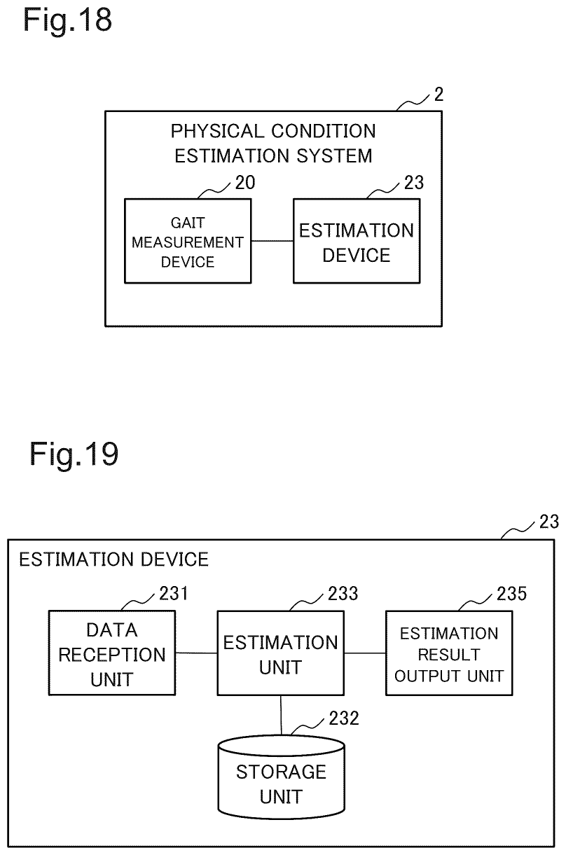

FIG. 18 is a block diagram illustrating an example of a configuration of a physical condition estimation system 2 according to the present example embodiment. The physical condition estimation system 2 includes a gait measurement device 20 and an estimation device 23. In the present example embodiment, an example in which the gait measurement device 20 and the estimation device 23 are configured as separate pieces of hardware will be described. For example, the gait measurement device 20 is installed on footwear or the like of a subject (a user) who is a target in estimating a physical condition. For example, the function of the estimation device 23 is installed in a mobile terminal carried by the subject (user). The gait measurement device 20 has the same configuration as the gait measurement device 10 according to the first example embodiment. Hereinafter, the description of the gait measurement device 20 will be omitted, and the estimation device 23 will be mainly described.

### [Estimation Device]

FIG. 19 is a block diagram illustrating an example of a configuration of the estimation device 23. The estimation device 23 includes a data reception unit 231, a storage unit 232, an estimation unit 233, and an estimation result output unit 235.

The data reception unit 231 receives feature quantity data from the gait measurement device 20. The data reception unit 231 outputs the received feature quantity data to the estimation unit 233. The data reception unit 231 may receive the feature quantity data from the gait measurement device 20 via a wire such as a cable, or may receive the feature quantity data from the gait measurement device 20 through wireless communication. For example, the data reception unit 231 is configured to receive the feature quantity data from the gait measurement device 20 through a wireless communication function (not illustrated) conforming to a standard such as Bluetooth (registered trademark) or WiFi (registered trademark). Note that the communication function of the data reception unit 231 may conform to a standard other than Bluetooth (registered trademark) or WiFi (registered trademark).

The storage unit 232 stores an estimation model that estimates a physical condition of an estimation target using the feature quantity data extracted from the gait waveform data. The storage unit 232 stores an estimation model that has learned a plurality of subjects. For example, the storage unit 232 stores an estimation model that has learned a plurality of subjects to estimate a physical condition. The estimation model may be stored in the storage unit 232 at the time of shipping a product to a factory, calibration before the user uses the physical condition estimation system, or the like. Note that, in a case where an estimation model stored in a storage device such as an external server is used, the estimation model may be used via an interface (not illustrated) connected to the storage device. In that case, an estimation model that estimates a physical condition may not be stored in the storage unit 232.

The estimation unit 233 acquires the feature quantity data from the data reception unit 231. The estimation unit 233 estimates a physical condition of an estimation target using the acquired feature quantity data. The estimation unit 233 inputs the feature quantity data to the estimation model stored in the storage unit 232. The estimation unit 233 outputs an estimation result according to an output (estimated value) from the estimation model. In a case where an estimation model stored in an external storage device constructed in a cloud, a server, or the like is used, the estimation model may be used via an interface (not illustrated) connected to the storage device.

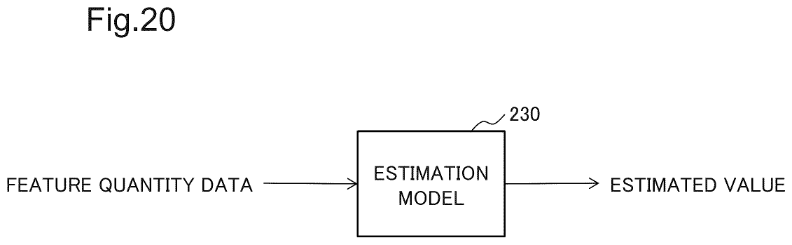

FIG. 20 is a conceptual diagram illustrating an example in which an estimated value is output by inputting feature quantity data corresponding to sensor data measured according to a gait of a user to the estimation model 230 constructed in advance for estimating a physical condition of an estimation target. For example, in a case where the estimation model 230 estimates a first metatarsophalangeal angle FMTPA, a first metatarsophalangeal angle FMTPA is output from the estimation model 230 as feature quantity data is input thereto. For example, in a case where the estimation model 230 estimates a center of pressure excursion index CPEI, a center of pressure excursion index CPEI is output from the estimation model 230 as feature quantity data is input thereto. If an estimation result regarding a physical feature is output in response to the input of the feature quantity data of the gait phase clusters, the estimation result estimated by the estimation model 230 is not limited.

For example, the estimation unit 233 estimates a physical condition of an estimation target using the multiple regression prediction method. For example, the estimation unit 233 estimates a first metatarsophalangeal angle FMTPA using the following Formula 1.

$$FMTPA = β1 \times C 1 + β2 \times C 2 + … + β11 \times C 11 + β0 \qquad ( 1 )$$

In the above Formula 1, C1, C2, . . . , and C11 are feature quantities for the respective gait phase clusters to be used in estimating a first metatarsophalangeal angle FMTPA shown in the correspondence table of FIG. 11. β1, β2, . . . , and β11 are coefficients to be multiplied by C1, C2, . . . , and C11. β0 is a constant term. For example, the coefficients such as β1, β2, . . . , and β11 and the constant term β0 are stored in the storage unit 232.

In the above Formulas 1 and 2, a value of a feature quantity of a gait phase cluster having a small number of components is sufficiently smaller than those of the other gait phase clusters. Therefore, a coefficient to be multiplied by the feature quantity of the gait phase cluster having a small number of components is set to a value larger than coefficients to be multiplied by the feature quantities of the other gait phase clusters. For example, in the above Formula 1, β1 is set to about −100, β2 is set to about 3000, and the other coefficients are set to 20 or less.

A sudden variation in feature quantity of a gait phase cluster having a small number of components becomes a factor that greatly varies an estimated value of a physical condition. In the present example embodiment, at the time of selecting a feature quantity by the gait measurement device 20, a feature quantity that can be a factor that varies an estimated value is removed. Therefore, the method according to the present example embodiment is hardly affected by a variation in feature quantity of a gait phase cluster having a small number of components. Since the feature quantity of the gait phase cluster having a small number of components is removed, there is a possibility that the accuracy is lowered in estimating a physical condition of an estimation target. However, if the number of gait phase clusters, from which a plurality of feature quantities constituting the feature quantity data are extracted, is large, a decrease in estimation accuracy resulting from the removal of the feature quantity of the gait phase cluster having a small number of components can be ignored.

The estimation result output unit 235 outputs a physical condition estimation result of the estimation unit 233. For example, the estimation result output unit 235 displays a physical condition estimation result on a screen of a mobile terminal of a subject (user). For example, the estimation result output unit 235 outputs an estimation result to an external system or the like that uses the estimation result.

The use of the feature quantity data of the gait phase cluster output from the estimation device 23 is not particularly limited. For example, the estimation device 23 is connected to an external system or the like constructed in a cloud or a server via a mobile terminal (not illustrated) carried by the subject (user). The mobile terminal (not illustrated) is a portable communication device. For example, the mobile terminal is a portable communication device having a communication function, such as a smartphone, a smart watch, or a mobile phone. For example, the estimation device 23 is connected to the mobile terminal via a wire such as a cable. For example, the estimation device 23 is connected to the mobile terminal through wireless communication. For example, the estimation device 23 is connected to the mobile terminal through a wireless communication function (not illustrated) conforming to a standard such as Bluetooth (registered trademark) or WiFi (registered trademark). Note that the communication function of the estimation device 23 may conform to a standard other than Bluetooth (registered trademark) or WiFi (registered trademark). A physical condition estimation result may be used by an application installed in the mobile terminal. In that case, the mobile terminal executes processing using the estimation result by application software or the like installed in the mobile terminal.

### (Operation)

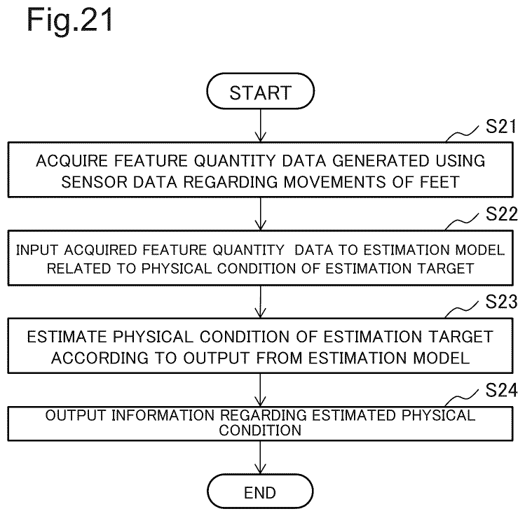

Next, an operation of the physical condition estimation system 2 will be described with reference to the drawings. Here, an operation of the estimation device 23 included in the physical condition estimation system 2 will be described. FIG. 21 is a flowchart for explaining the operation of the estimation device 23. In the description based on the flowchart of FIG. 21, the estimation device 23 will be described as an operation subject.

In FIG. 21, first, the estimation device 23 acquires feature quantity data generated using sensor data regarding movements of feet (step S21).

Next, the estimation device 23 inputs the acquired feature quantity data to the estimation model 230 that estimates a physical condition of an estimation target (step S22).

Next, the estimation device 23 estimates a physical condition of an estimation target according to the output (estimated value) from the estimation model 230 (step S23).

Next, the estimation device 23 outputs information regarding the estimated physical condition (step S24).

### [Example of Application]

Next, an example of application according to the present example embodiment will be described with reference to the drawings. In the following example of application, an example in which the estimation device 23 installed in the mobile terminal carried by the user estimates a physical condition using feature quantity data measured by the gait measurement devices 20 arranged in shoes will be described.

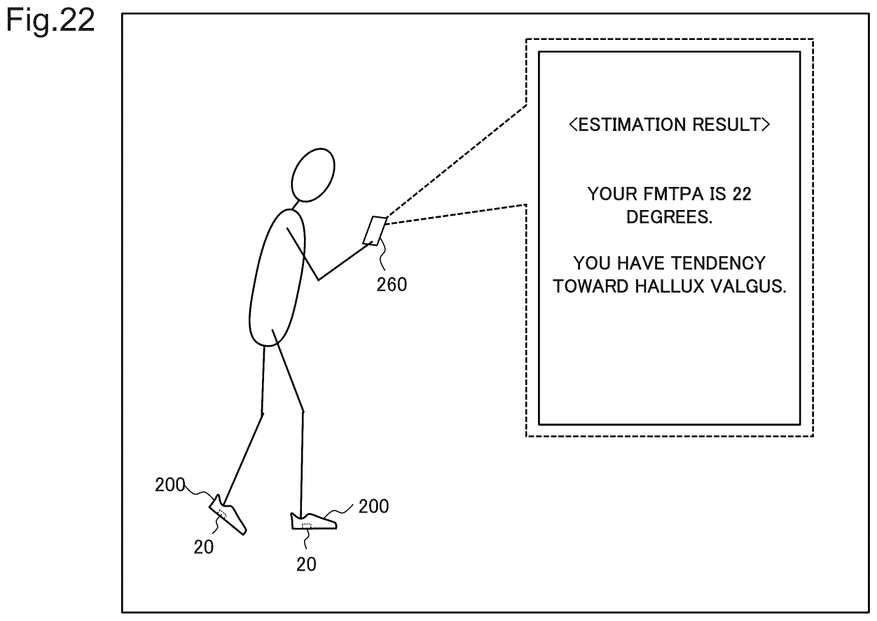

FIG. 22 is a conceptual diagram illustrating an example in which a result of estimation by the estimation device 23 is displayed on a screen of a mobile terminal 260 carried by the user who walks while wearing shoes 200 in which the gait measurement devices 20 are arranged. FIG. 22 illustrates an example in which information related to an estimation result using feature quantity data corresponding to sensor data measured while the user is walking is displayed on the screen of the mobile terminal 260.

FIG. 22 illustrates an example in which a degree of progress of hallux valgus corresponding to a size of a first metatarsophalangeal angle (FMTPA) is displayed on the screen of the mobile terminal 260. In the example of FIG. 22, based on the feature quantity data including feature quantities extracted from the sensor data measured while the user is walking, the information “Your FMTPA is 22 degrees. You have a tendency toward hallux valgus.” is displayed on the display unit of the mobile terminal 260. The user who has confirmed the information displayed on the display unit of the mobile terminal 260 can recognize the degree of progress of his/her hallux valgus. For example, in a case where the degree of progress of hallux valgus is high, a message recommending an examination in a hospital or contact information of an appropriate hospital may be displayed on the display unit of the mobile terminal 260.

FIG. 20 is an example, and does not limit a method of using the result of estimation by the estimation device 23 according to the present example embodiment. For example, information regarding a gait such as degrees of pronation/supination of the left and right feet, step lengths of the left and right feet, a trajectory of spinning, symmetry, and a foot angle may be displayed on the screen of the mobile terminal 260.

As described above, the physical condition estimation system according to the present example embodiment includes a gait measurement device and an estimation device. The gait measurement device acquires time-series data of sensor data regarding movements of feet. The gait measurement device extracts gait waveform data for one gait cycle from the time-series data of the sensor data, and normalizes the extracted gait waveform data. The gait measurement device extracts, from the normalized gait waveform data, feature quantities related to a physical condition of an estimation target from gait phase clusters each constituted by one or more temporally consecutive gait phases. The gait measurement device selects feature quantities to be used in estimating the physical condition from the extracted feature quantities for the respective gait phase clusters using a preset threshold as a reference. The gait measurement device generates feature quantity data including the selected feature quantities. The gait measurement device outputs the generated feature quantity data to the estimation device.

The estimation device estimates a physical condition of an estimation target related to the user wearing the footwear in which the gait measurement device is installed using the feature quantity data output from the gait measurement device. For example, the estimation device inputs the feature quantity data output from the gait measurement device to an estimation model, and estimates the physical condition of the user according to an output from the estimation model. For example, the estimation model is a model obtained by learning teacher data in which a feature quantity extracted from a gait phase cluster in which a feature related to the physical condition of the estimation target appears is an explanatory variable and a value corresponding to the physical condition of the estimation target is an objective variable.

The physical condition estimation system according to the present example embodiment estimates a physical condition of a user using feature quantity data measured by the gait measurement device in such a way that a physical condition can be estimated with high accuracy. Therefore, according to the present aspect, it is possible to estimate a physical condition with high accuracy.

### Third Example Embodiment

Next, a machine learning system according to a third example embodiment will be described with reference to the drawings. The machine learning system according to the present example embodiment generates an estimation model for estimating a physical condition according to an input of a feature quantity by machine learning using feature quantity data extracted from sensor data measured by the gait measurement device.

### (Configuration)

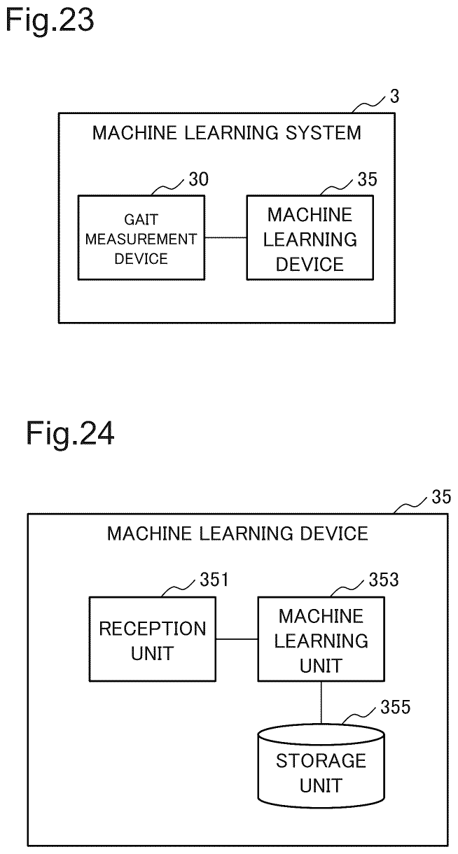

FIG. 23 is a block diagram illustrating an example of a configuration of a machine learning system 3 according to the present example embodiment. The machine learning system 3 includes a gait measurement device 30 and a machine learning device 35. The gait measurement device 30 and the machine learning device 35 may be connected to each other in a wired or wireless manner. The gait measurement device 30 and the machine learning device 35 may be constituted by a single device. In addition, the machine learning system 3 may be constituted only by the machine learning device 35 except the gait measurement device 30 from the configuration of the machine learning system 3. Although only one gait measurement device 30 is illustrated in FIG. 23, gait measurement devices (two in total) 30 may be arranged on left and right feet, respectively. Furthermore, the machine learning device 35 may not be connected to the gait measurement device 30, and may be configured to execute machine learning using feature quantity data generated in advance by the gait measurement device 30 and stored in a database.

The gait measurement device 30 is installed on at least one of the left and right feet. The gait measurement device 30 has the same configuration as the gait measurement device 10 according to the first example embodiment. The gait measurement device 30 includes an acceleration sensor and an angular velocity sensor. The gait measurement device 30 converts a measured physical quantity into digital data (also referred to as sensor data). The gait measurement device 30 generates normalized gait waveform data for one gait cycle from the time-series data of the sensor data. The gait measurement device 30 generates feature quantity data to be used in estimating a physical condition of an estimation target. The gait measurement device 30 transmits the generated feature quantity data to the machine learning device 35. Note that the gait measurement device 30 may be configured to transmit the feature quantity data to a database (not illustrated) accessed by the machine learning device 35. The feature quantity data accumulated in the database is used for machine learning by the machine learning device 35.

The machine learning device 35 receives the feature quantity data from the gait measurement device 30. In a case where the feature quantity data accumulated in the database (not illustrated) is used, the machine learning device 35 receives the feature quantity data from the database. The machine learning device 35 executes machine learning using the received feature quantity data. For example, the machine learning device 35 learns, as teacher data, feature quantity data extracted from gait waveform data for a plurality of subjects and a value related to a physical condition of an estimation target according to the feature quantity data. An algorithm for the machine learning executed by the machine learning device 35 is not particularly limited. The machine learning device 35 generates an estimation model that has learned a plurality of subjects. The machine learning device 35 stores the generated estimation model. The estimation model trained by the machine learning device 35 may be stored in a storage device outside the machine learning device 35.

### [Machine Learning Device]

Next, the machine learning device 35 will be described in detail with reference to the drawings. FIG. 24 is a block diagram illustrating an example of a detailed configuration of the machine learning device 35. The machine learning device 35 includes a reception unit 351, a machine learning unit 353, and a storage unit 355.

The reception unit 351 receives feature quantity data from the gait measurement device 30. The reception unit 351 outputs the received feature quantity data to the machine learning unit 353. The reception unit 351 may receive the feature quantity data from the gait measurement device 30 via a wire such as a cable, or may receive the feature quantity data from the gait measurement device 30 through wireless communication. For example, the reception unit 351 is configured to receive the feature quantity data from the gait measurement device 30 through a wireless communication function (not illustrated) conforming to a standard such as Bluetooth (registered trademark) or WiFi (registered trademark). Note that the communication function of the reception unit 351 may conform to a standard other than Bluetooth (registered trademark) or WiFi (registered trademark).

The machine learning unit 353 acquires the feature quantity data from the reception unit 351. The machine learning unit 353 executes machine learning using the acquired feature quantity data. For example, the machine learning unit 353 learns, as teacher data, a data set in which a feature quantity extracted from a user whose certain physical condition is measured is an explanatory variable and the physical condition of the user is an objective variable. For example, the machine learning unit 353 generates an estimation model that has learned a plurality of users to estimate a physical condition based on feature quantity data. For example, the machine learning unit 353 stores an estimation model that has learned a plurality of users in the storage unit 355.

For example, the machine learning unit 353 executes machine learning using a linear regression algorithm. For example, the machine learning unit 353 executes machine learning using a support vector machine (SVM) algorithm. For example, the machine learning unit 353 executes machine learning using a Gaussian process regression (GPR) algorithm. For example, the machine learning unit 353 executes machine learning using a random forest (RF) algorithm. For example, the machine learning unit 353 may execute unsupervised machine learning for classifying a user who is a source from which feature quantity data is generated, according to the feature quantity data. The algorithm for the machine learning executed by the machine learning unit 353 is not particularly limited.

The machine learning unit 353 may execute machine learning using gait waveform data for one gait cycle as an explanatory variable. For example, the machine learning unit 353 executes supervised learning in which gait waveform data on accelerations in the three-axis directions, angular velocities around the three axes, and angles (attitude angles) around the three axes is an explanatory variable and a correct value of a physical condition of an estimation target is an objective variable. For example, in a case where the gait phase is set in 1% increments in a gait cycle of 0 to 100%, the machine learning unit 353 performs machine learning using 909 explanatory variables.

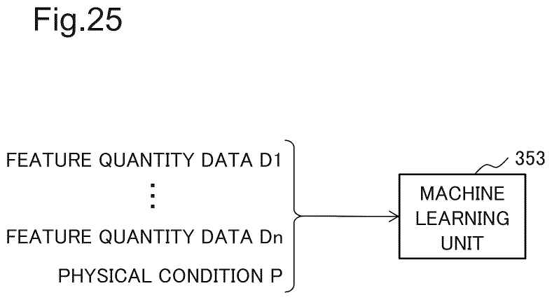

FIG. 25 is a conceptual diagram illustrating an example in which the machine learning unit 353 learns, as teacher data, a data set of feature quantity data D1 to Dn as explanatory variables and a physical condition P as an objective variable (n is a natural number). For example, the machine learning unit 353 generates an estimation model that learns data for a plurality of subjects, and outputs an output (estimated value) related to a physical condition of an estimation target according to an input of a feature quantity extracted from sensor data.

The storage unit 355 stores an estimation model that has learned a plurality of subjects. For example, the storage unit 355 stores an estimation model that has learned a plurality of subjects to estimate a physical condition of an estimation target. For example, the estimation model stored in the storage unit 355 is used by the estimation device 23 according to the second example embodiment to estimate a physical condition.

As described above, the machine learning system according to the present example embodiment includes a gait measurement device and a machine learning device. The gait measurement device acquires time-series data of sensor data regarding movements of feet. The gait measurement device extracts gait waveform data for one gait cycle from the time-series data of the sensor data, and normalizes the extracted gait waveform data. The gait measurement device extracts, from the normalized gait waveform data, feature quantities related to a physical condition of an estimation target from gait phase clusters each constituted by one or more temporally consecutive gait phases. The gait measurement device selects feature quantities to be used in estimating the physical condition from the extracted feature quantities for the respective gait phase clusters using a preset threshold as a reference. The gait measurement device generates feature quantity data including the selected feature quantities. The gait measurement device outputs the generated feature quantity data to the machine learning device.

The machine learning device includes a reception unit, a machine learning unit, and a storage unit. The reception unit acquires feature quantity data generated by the gait measurement device. The machine learning unit executes machine learning using the feature quantity data. The machine learning unit generates an estimation model that outputs a physical condition according to an input of a feature quantity (second feature quantity) of a gait phase cluster extracted from the time-series data of the sensor data measured according to a gait of the user. For example, the machine learning unit generates an estimation model that outputs a degree of hallux valgus (first metatarsophalangeal angle FMTPA) according to an input of a feature quantity (second feature quantity) of a gait phase cluster extracted from the time-series data of the sensor data measured according to a gait of the user. The estimation model generated by the machine learning unit is stored in the storage unit.

The machine learning system according to the present example embodiment generates an estimation model using feature quantity data measured by the gait measurement device in such a way that a physical condition can be estimated with high accuracy. Therefore, according to the present aspect, it is possible to generate an estimation model capable of estimating a physical condition with high accuracy.

### Fourth Example Embodiment

Next, a feature quantity data generation device according to a fourth example embodiment will be described with reference to the drawings. The feature quantity data generation device according to the present example embodiment has a configuration in which the feature quantity data generation unit included in the gait measurement device according to each of the first to third example embodiments is simplified.

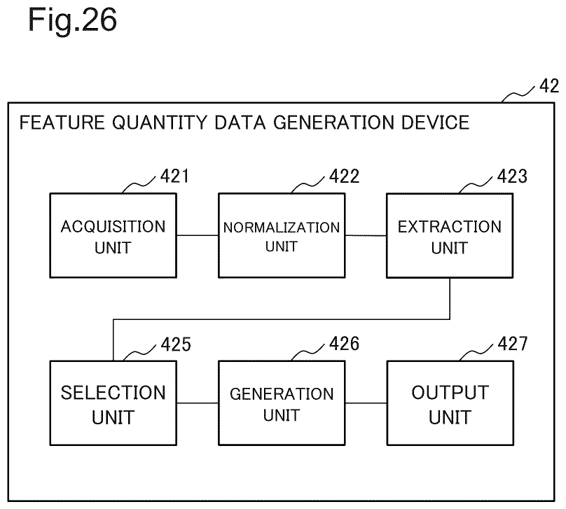

FIG. 26 is a block diagram illustrating an example of a configuration of a feature quantity data generation device 42 according to the present example embodiment. The feature quantity data generation device 42 includes an acquisition unit 421, a normalization unit 422, an extraction unit 423, a selection unit 425, a generation unit 426, and an output unit 427.

The acquisition unit 421 acquires time-series data of sensor data regarding movements of feet. The normalization unit 422 extracts gait waveform data for one gait cycle from the time-series data of the sensor data, and normalizes the extracted gait waveform data. The extraction unit 423 extracts, from the normalized gait waveform data, feature quantities related to a physical condition of an estimation target from gait phase clusters each constituted by one or more temporally consecutive gait phases. The selection unit 425 selects feature quantities to be used in estimating the physical condition from the extracted feature quantities for the respective gait phase clusters using a preset threshold as a reference. The generation unit 426 generates feature quantity data including the selected feature quantities. The output unit 427 outputs the generated feature quantity data.

As described above, in the present example embodiment, a gait waveform for one gait cycle is normalized, and feature quantities to be used in estimating a physical condition are selected using a preset threshold as a reference. Therefore, according to the present example embodiment, it is possible to generate feature quantity data with which a physical condition can be estimated with high accuracy.

### (Hardware)

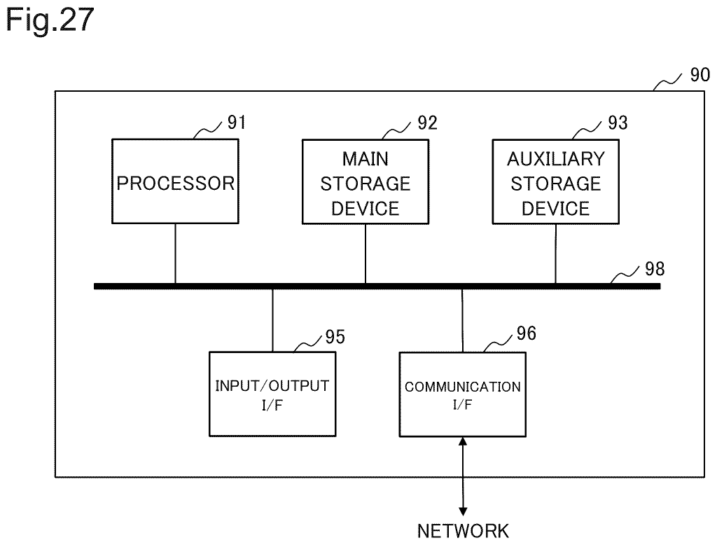

Here, a hardware configuration for executing the control or processing according to each of the above-described example embodiments of the present disclosure will be described using an information processing device 90 illustrated in FIG. 27 as an example. Note that the information processing device 90 of FIG. 27 is an example of the configuration for executing the control or processing according to each of the above-described example embodiments, and does not limit the scope of the present disclosure.

As illustrated in FIG. 27, the information processing device 90 includes a processor 91, a main storage device 92, an auxiliary storage device 93, an input/output interface 95, and a communication interface 96. In FIG. 27, an interface is abbreviated as an I/F. The processor 91, the main storage device 92, the auxiliary storage device 93, the input/output interface 95, and the communication interface 96 are connected to each other via a bus 98 for data communication therebetween. In addition, the processor 91, the main storage device 92, the auxiliary storage device 93, and the input/output interface 95 are connected to a network such as the Internet or an intranet via the communication interface 96.

The processor 91 develops a program stored in the auxiliary storage device 93 or the like in the main storage device 92. The processor 91 executes the program developed in the main storage device 92. In the present example embodiment, a software program installed in the information processing device 90 may be used. The processor 91 executes the control or processing according to each of the above-described example embodiments.

The main storage device 92 has an area in which a program is developed. A program stored in the auxiliary storage device 93 or the like is developed in the main storage device 92 by the processor 91. The main storage device 92 is achieved by, for example, a volatile memory such as a dynamic random access memory (DRAM). In addition, a nonvolatile memory such as a magnetoresistive random access memory (MRAM) may be included/added as the main storage device 92.

The auxiliary storage device 93 stores various data such as programs. The auxiliary storage device 93 is achieved by a local disk such as a hard disk or a flash memory. Note that various data may be stored in the main storage device 92, and the auxiliary storage device 93 may be omitted.

The input/output interface 95 is an interface for connecting the information processing device 90 and a peripheral device to each other in accordance with a standard or a specification. The communication interface 96 is an interface for connection to an external system or device through a network such as the Internet or an intranet in accordance with a standard or a specification. The input/output interface 95 and the communication interface 96 may be constituted by a common interface connected to an external device.

An input device such as a keyboard, a mouse, or a touch panel may be connected to the information processing device 90 if necessary. These input devices are used to input information and settings. In a case where the touch panel is used as an input device, a display screen of a display device may also serve as an interface of the input device. Data communication between the processor 91 and the input device may be mediated by the input/output interface 95.

Furthermore, the information processing device 90 may include a display device for displaying information. In a case where the information processing device 90 includes a display device, the information processing device 90 preferably includes a display control device (not illustrated) for controlling the display of the display device. The display device may be connected to the information processing device 90 via the input/output interface 95.

Furthermore, the information processing device 90 may be equipped with a drive device. Between the processor 91 and the recording medium (program recording medium), the drive device mediates reading of data or a program from the recording medium, writing of a processing result of the information processing device 90 to the recording medium, and the like. The drive device only needs to be connected to the information processing device 90 via the input/output interface 95.

An example of the hardware configuration for enabling the control or processing according to each of the above-described example embodiments of the present disclosure has been described above. Note that the hardware configuration of FIG. 27 is an example of the hardware configuration for executing the control or processing according to each of the above-described example embodiments, and does not limit the scope of the present disclosure. In addition, a program for causing a computer to execute the control or processing according to each of the above-described example embodiments also falls within the scope of the present disclosure. Furthermore, a program recording medium recording the program according to each of the above-described example embodiments also falls within the scope of the present disclosure. The recording medium can be achieved by, for example, an optical recording medium such as a compact disc (CD) or a digital versatile disc (DVD). The recording medium may be achieved by a semiconductor recording medium such as a universal serial bus (USB) memory or a secure digital (SD) card. Furthermore, the recording medium may be achieved by a magnetic recording medium such as a flexible disk, or another recording medium. In a case where a program executed by the processor is recorded in the recording medium, the recording medium is a program recording medium.

The components of the above-described example embodiments may be combined in any manner. In addition, the components according to each of the above-described example embodiments may be achieved by software or by a circuit.

While the invention has been particularly shown and described with reference to example embodiments thereof, the invention is not limited to these embodiments. It will be understood by those of ordinary skill in the art that various changes in form and details may be made therein without departing from the spirit and scope of the present invention as defined by the claims.

### REFERENCE SIGNS LIST

2 Physical condition estimation system
3 Machine learning system
10, 20, 30 Gait measurement device
11 Sensor
12 Feature quantity data generation unit
23 Estimation device
35 Machine learning device
42 Feature quantity data generation device
111 Acceleration sensor
112 Angular velocity sensor
121, 421 Acquisition unit
122, 422 Normalization unit
123, 423 Extraction unit
125, 425 Selection unit
126, 426 Generation unit
127, 427 Output unit
230 Estimation model
231 Data reception unit
232 Storage unit
233 Estimation unit
235 Estimation result output unit
351 Reception unit
353 Machine learning unit
355 Storage unit

What is claimed is:

1. A feature quantity data generation device comprising:
a memory storing instructions; and
a processor connected to the memory and configured to execute the instructions to:
acquire time-series data of sensor data regarding movements of feet;
extract gait waveform data for one gait cycle from the time-series data of the sensor data, and normalize the extracted gait waveform data;
extract, from the normalized gait waveform data, feature quantities related to a physical condition of an estimation target from gait phase clusters each constituted by one or more temporally consecutive gait phases;
select feature quantities to be used in estimating the physical condition from the extracted feature quantities for the respective gait phase clusters using a preset threshold as a reference;
generate feature quantity data including the selected feature quantities; and
output the generated feature quantity data.

2. The feature quantity data generation device according to claim 1, wherein
in response to a value of any of the extracted feature quantities of the gait phase clusters exceeding a variation threshold set in advance for each of the feature quantities of the gait phase clusters,
the processor is configured to execute the instructions to
remove the feature quantity exceeding the variation threshold.

3. The feature quantity data generation device according to claim 1, wherein
in response to a value of any of the extracted feature quantities of the gait phase clusters exceeding a variation threshold set in advance for each of the feature quantities of the gait phase clusters,
the processor is configured to execute the instructions to
scan feature quantities of the gait phases before and after the gait phase constituting the gait phase cluster with feature quantities exceeding the variation threshold, and
select a feature quantity lower than the variation threshold.

4. The feature quantity data generation device according to claim 1, wherein
the processor is configured to execute the instructions to
select feature quantities of gait phase clusters in each of which the number of components that are gait phases constituting the gait phase cluster exceeds a selection threshold.

5. The feature quantity data generation device according to claim 1, wherein
the processor is configured to execute the instructions to
detect a timing of a heel contact and a timing of a toe off from the time-series data of the sensor data,
extract a section between consecutive heel contacts as the gait waveform data for one gait cycle,
execute first normalization in which the gait cycle of the gait waveform data is set in such a way that the preceding heel contact is 0% and the following heel contact is 100%, and
execute second normalization in which a section between the preceding heel contact and the toe off is 60% and a section between the toe off and the following heel contact is 40%.

6. A gait measurement device comprising:
the feature quantity data generation device according to claim 1; and
a sensor installed in footwear of a user who is a target in estimating a physical condition, and configured to measure spatial accelerations and spatial angular velocities, generate sensor data regarding movements of feet using the measured spatial accelerations and the spatial angular velocities, and transmit the generated sensor data to the feature quantity data generation device.

7. A physical condition estimation system comprising:
the gait measurement device according to claim 6; and
an estimation device that comprises
a memory storing instructions; and
a processor connected to the memory and configured to execute the instructions to
estimate a physical condition of an estimation target related to the user wearing the footwear in which the gait measurement device is installed using the feature quantity data output from the gait measurement device.

8. The physical condition estimation system according to claim 7, wherein
the processor of the estimation device is configured to execute the instructions to input the feature quantity data output from the gait measurement device to an estimation model, and
estimate the physical condition of the user according to an output from the estimation model, the estimation model being obtained by machine learning teacher data in which a feature quantity extracted from a gait phase cluster in which a feature related to the physical condition of the estimation target appears is an explanatory variable and a value corresponding to the physical condition of the estimation target is an objective variable.

9. A feature quantity data generation method performed by a computer, the method comprising:
acquiring time-series data of sensor data regarding movements of feet;
extracting gait waveform data for one gait cycle from the time-series data of the sensor data;
normalizing the extracted gait waveform data;
extracting, from the normalized gait waveform data, feature quantities related to a physical condition of an estimation target from gait phase clusters each constituted by one or more temporally consecutive gait phases;
selecting feature quantities to be used in estimating the physical condition from the extracted feature quantities for the respective gait phase clusters using a preset threshold as a reference;
generating feature quantity data including the selected feature quantities; and
outputting the generated feature quantity data.

10. A non-transitory recording medium recording a program for causing a computer to execute:
processing of acquiring time-series data of sensor data regarding movements of feet;
processing of extracting gait waveform data for one gait cycle from the time-series data of the sensor data;
processing of normalizing the extracted gait waveform data;
processing of extracting, from the normalized gait waveform data, feature quantities related to a physical condition of an estimation target from gait phase clusters each constituted by one or more temporally consecutive gait phases;
processing of selecting feature quantities to be used in estimating the physical condition from the extracted feature quantities for the respective gait phase clusters using a preset threshold as a reference;
processing of generating feature quantity data including the selected feature quantities; and
processing of outputting the generated feature quantity data.

11. The feature quantity data generation device according to claim 1, wherein
the feature quantity data is configured to be used to make a decision concerning healthcare.
